(19) United States
(12) Patent Application Publication
(10) Pub. No.: US 2026/0114449 A1
(43) Pub. Date: Apr. 30, 2026

(54) CONCENTRATED ANTIMICROBIAL SOLUTIONS CONTAINING SPARINGLY SOLUBLE ANTIMICROBIAL AGENTS

(71) Applicant: Virox Technologies Inc., Oakville (CA)

(72) Inventor: Faraz Ahmadpour ALDERSON, Mississauga (CA)

(73) Assignee: Virox Technologies Inc., Oakville, ON (CA)

(21) Appl. No.: 19/117,790

(22) PCT Filed: Sep. 29, 2023
(86) PCT No.: PCT/IB2023/059809

§ 371 Date: Apr. 2, 2025

(51) Int. Cl. A01N 37/40; A01N 25/04; A01N 31/08; A01N 37/10; A01N 41/04; A01P 1/00

(52) U.S. Cl. CPC A01N 37/40; A01N 25/04; A01N 31/08; A01N 37/10; A01N 41/04; A01P 1/00

(57) ABSTRACT

A phase stable, concentrated antimicrobial solution having a pH less than 0.6, comprising:
a. an effective amount of at least one sparingly soluble antimicrobial agent;
b. an effective amount of at least one antimicrobial surfactant selected from the group consisting of anionic surfactants and amphoteric surfactants; and
c. an effective amount of at least one solvent selected from the group consisting of esteramides, diamides, branched dibasic esters, and alkyl pyrrolidones;
wherein, the weight ratio of the at least one antimicrobial surfactant to the at least one solvent is from about 1:3 to about 3:1, and wherein the solution, when diluted with an aqueous diluent at a ratio of from about 1:64 to about 1:1024 has a pH of from about 1 to about 6, and passes the Germicidal Spray Testing AOAC 961.02 test method, at room temperature, and at a contact time of no greater than 10 minutes against S. aureus. Effective ready-to-use (RTU) solutions, kits for use in making the concentrated and RTU solutions, methods of antimicrobial reduction using the solutions, and methods of making the solutions are also disclosed.

### CROSS-REFERENCE TO RELATED APPLICATIONS

This application claims priority from U.S. provisional application 63/412,975 filed Oct. 4, 2022, the entire contents of which are incorporated herein by reference.

### TECHNICAL FIELD

The present invention relates to antimicrobial solutions for sterilization, disinfection or sanitization of surfaces and, more particularly, to concentrated antimicrobial solutions that are effective upon dilution.

### BACKGROUND

Antimicrobial solutions, such as sterilants, disinfectants, and sanitizers, are used to kill microbes on surfaces for infection control and prevention. It is desirable to formulate concentrated versions that are to be diluted by the end user prior to use to save costs in manufacturing, shipping, and storage, and to reduce their carbon footprint.

A challenge exists in formulating antimicrobial solutions to optimize their desirable attributes in concentrated and diluted ready-to-use (RTU) forms. These desirable attributes include product safety, no or low toxicity, broad spectrum of activity, no or low objectionable odor, an acceptable shelf-life at room temperature for at least one year (preferably at least two years), surface compatibility (i.e., non-corrosive or non-damaging to surfaces), and effective cleaning properties so as to be effective to kill microbes on surfaces contaminated with a soil load.

Furthermore, concentrated antimicrobial solutions should be in a single phase, i.e., the ingredients should not precipitate under normal storage conditions and during a normal shelf-life, so that RTU solutions can be made that are consistent from one batch to another. They also need to be effective upon dilution at reasonable contact times (e.g., 10 minutes or less, based on the United States Environmental Protection Act's requirements for surface disinfectants).

Antimicrobial solutions contain antimicrobial agents which are ingredients or compounds that function to kill microbes. The agents that can be formulated into a concentrated product include aldehydes (such as glutaraldehyde and phthalaldehyde), quaternary ammonium compounds (also known as “quats”), short chain linear alkyl hydroxy carboxylic acids (e.g., lactic acid, glycolic acid), chlorine bleaches (such as sodium hypochlorite), peroxycarboxylic acids (such as peracetic acid (PAA)), and other peroxide compounds (such as hydrogen peroxide). However, each of these classes of antimicrobial agents have drawbacks. For example, aldehydes and quats are considered toxic at certain concentrations, and quats have also been associated with microbial resistance. Short chain linear or branched carboxylic acids need to be present in relatively higher concentrations in the RTU solution to deliver the needed breadth and speed of killing microbes, which limits the degree to which these solutions can be concentrated. Oxidizers such as chlorine bleaches, peroxycarboxylic acids, and other peroxide compounds can be hazardous, e.g., corrosive and/or explosive, when formulated as concentrates. Some of these compounds (e.g., chlorine bleach and PAA) are noxious or malodorous in both concentrated and RTU forms, and others (e.g., hydrogen peroxide) need to be stabilized in order to provide a product with an adequate shelf-life. Quats and chlorine bleach can also irritate the lungs and skin, even at relatively low concentrations.

There are several antimicrobial agents that do not suffer from the above drawbacks but are difficult to formulate into concentrated solutions because of their low solubility in water. These antimicrobial agents include aromatic carboxylic acids (such as salicylic acid and benzoic acid), essential oils, terpenes, terpenoids, and phenolic compounds. Examples of essential oils, terpenes and terpenoids include thymol, linalool, limonene, cinnamaldehyde, eugenol, geraniol, neem oil, and carvacrol. Examples of phenolic compounds include salicylic acid, hydroxyacetophenone, and menthol.

There is an ongoing need to provide a safe, economical, user-friendly, and environmentally friendly concentrated antimicrobial solution that can be diluted at high ratios while still providing an efficacious RTU solution. The present invention is intended to meet this need.

### SUMMARY OF THE INVENTION

According to a first aspect, the invention provides a phase stable, concentrated antimicrobial solution having a pH less than 0.6, comprising, consisting essentially of, or consisting of:
a. an effective amount of at least one sparingly soluble antimicrobial agent;
b. an effective amount of at least one antimicrobial surfactant selected from the group consisting of anionic surfactants and amphoteric surfactants; and
c. an effective amount of at least one solvent selected from the group consisting of esteramides, diamides, branched dibasic esters, and alkyl pyrrolidones;
wherein, the weight ratio of the at least one antimicrobial surfactant to the at least one solvent is from about 1:3 to about 3:1, and wherein the solution, when diluted with an aqueous diluent at a ratio of from about 1:64 to about 1:1024 has a pH of from about 1 to about 6, and passes the Germicidal Spray Testing AOAC 961.02 test method, at room temperature, and at a contact time of no greater than 10 minutes against S. aureus.

The at least one sparingly soluble antimicrobial agent can be selected from the group consisting of phenolic compounds, C5-C28 carboxylic acids and salts thereof, terpenes, terpenoids, and essential oils.

Furthermore, the C5-C28 carboxylic acids and salts thereof can be selected from the group consisting of hexanoic acid, isohexanoic acid, hexenoic acid, hydroxyhexanoic acid, heptanoic acid, isoheptanoic acid, hepteneoic acid, hydroxyheptanoic acid, octanoic acid, isooctanoic acid, octanoic acid, hydroxyoctanoic acid, decanoic acid, isodecanoic acid, hydroxydecanoic acid, dodecanoic acid, dodecenoic acid, isodecanoic acid, lauroyl-1-lactylic acid, lauroyl-2-lactylic acid, myristoyl-1-lactylic acid, myristoyl-2-lactylic acid, palmitoyl-1-lactylic acid, palmitoyl-2-lactylic acid, steroyl-2-lactylic acid, cocoyl sarcosine, myristoyl sarcosine, oleoyl sarcosine, C10-C28 alkyl ether carboxylic acids, benzoic acid, phenoxyacetic acid, phenylacetic acid, salicylic acid, hydroxybenzoic acid, dihydroxybenzoic acid, tropic acid, phthalic acid, terephthalic acid, methylbenzoic acid, anthranilic acid, furoic acid, cinnamic acid, hydroxycinnamic acid, picolinic acid, dipicolinic acid, pyridinedicarboxylic acid, pyridinecarboxylic acid, and hydratropic acid. In some embodiments, the C5-C28 carboxylic acids and salts thereof are selected from the group consisting of hexanoic acid, heptanoic acid, octanoic acid, nonanoic acid, decanoic acid, benzoic acid, phenoxyacetic acid, phenylacetic acid, salicylic acid, phthalic acid, furoic acid, and cinnamic acid. In some embodiments, the at least one sparingly soluble antimicrobial agent is selected from the group consisting of salicylic acid, benzoic acid, thymol, carvacrol, gallic acid, limonene, phthalic acid, furoic acid, cinnamic acid, pinene, eucalyptol, neem oil, hexanoic acid, heptanoic acid, octanoic acid, nonanoic acid, and decanoic acid.

The antimicrobial essential oils, terpenes and terpenoids can be selected from the group consisting of thymol, carvacrol, eugenol, linalool, linalyl acetate, citronellol, geraniol, cineol/eucalyptol, thujone, pinene, neral, borneol, terpenyl acetate, limonene, terpinene, methyl eugenol, acetyl eugenol, myrcene, terpinolene, terpineol, anisol, citronellal, neem oil and its extracts, tea tree oil and its extracts, neryl acetate, methyl cinnamate, geranyl acetate, and bergamottin. In some embodiments, the antimicrobial essential oils, terpenes and terpenoids are selected from the group consisting of thymol, carvacrol, eugenol, limonene, cinnamaldehyde, pinene, terpinene, terpinol, neem oil, tea tree oil, eucalyptus oil, eucalyptol, and anisole.

The antimicrobial phenolic compounds can be selected from the group consisting of salicylic acid, capryloyl salicylic acid, methylsalicylic acid, coumaric acid, ferulic acid, diferulic acid, sinapic acid, caffeic acid, protocatechuic acid, syringic acid, vanillic acid, gallic acid, anthranilic acid, ellagic acid, methyl phenol, dimethyl phenol, trimethyl phenol, diphenol, benzyloxy phenol, menthol, phenylphenol, hydroxyacetophenone, hydroxypropiophenone, hydroxyalkyl phenol, gingerol, naphthol, and naphthalenediol. In some embodiments, the antimicrobial phenolic compounds are selected from the group consisting of salicylic acid, capryloyl salicylic acid, caffeic acid, gallic acid, anthranilic acid, menthol, phenylphenol, and hydroxyacetophenone.

The antimicrobial anionic surfactant can be selected from the group consisting of C8-C28 sulfonates, C8-C28 sulfates, C8-C28 ether sulfates, C8-C28 ether sulfonates, C8-C28 olefin sulfonates, C8-C28 olefin sulfates, C6-C28 carboxylates, C6-C28 ether carboxylates, C8-C28 sulfonated carboxylates, C8-C28 phosphates, C8-C28 phosphonates, C8-C28 phosphate esters, C8-C28 taurates, C8-C24 amino acid based surfactants, and acid forms thereof.

The amphoteric surfactant can be selected from the group consisting of C8-C24 alkyl amine oxides, alkyl betaines, alkylamido sultaines, and amphocarboxylates.

The esteramides can be according to Formula I below:

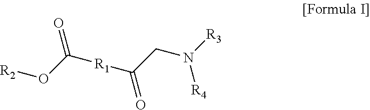

wherein R₁ is a C2-C8 branched or linear alkyl group;
R₂ is a C1-C6 linear, branched or cyclic alkyl group; and
R₃ and R₄ are independently methyl or ethyl.

Furthermore, the diamides can be according to Formula II:

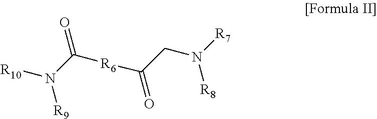

wherein R₆ is a C2-C8 branched or linear alkyl group; and
R₇, R₈, R₉ and R₁₀ are independently methyl or ethyl.

The alkyl pyrrolidones can be according to Formula III:

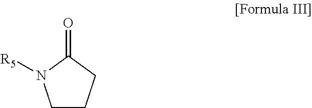

wherein R5 is a branched or unbranched, saturated or unsaturated, substituted or unsubstituted C2 to C6 alkyl chain. In some embodiments, R₅ is a branched or unbranched, saturated or unsaturated, unsubstituted C3 to C5 alkyl chain or a C3 alkyl chain substituted with a methoxy group.

The branched dibasic esters can be according to Formula IV below:

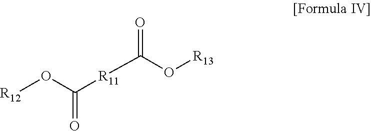

wherein R11 is a C2-C8 branched alkyl group; and
R12 and R13 are independently a C1-C4 linear or branched alkyl group.

In some embodiments, the at least one solvent is selected from the group consisting of N-butyl pyrrolidone, dimethyl 2-methylglutarate, pentanoic acid 5-(dimethylamino)-2-methyl-5-oxo-methyl ester and pentane-2-methyl-1,5-oxo-1,5-dimethylamine.

The at least one solvent and at least one antimicrobial surfactant function collectively to solubilize the at least one sparingly soluble antimicrobial agent. In some embodiments, the solubility of the at least one sparingly soluble antimicrobial agent is at least 20 times higher in the solution than in water. In some embodiments, the solubility of the at least one sparingly soluble antimicrobial agent is at least 30 times higher in the solution than in water. In some embodiments, the solubility of the at least one sparingly soluble antimicrobial agent is at least 40 times higher in the solution than in water. In some embodiments, the solubility of the at least one sparingly soluble antimicrobial agent is at least 50 times higher in the solution than in water. In some embodiments, the solubility of the at least one sparingly soluble antimicrobial agent is at least 60 times higher in the solution than in water.

The at least one antimicrobial surfactant and the at least one solvent are present at a combined total concentration that is enough to enhance the solubility of the at least one sparingly soluble antimicrobial agent as compared to its solubility in water. In some embodiments, this combined total is from at least 25 wt. %. In the same or other embodiments, this combined total is from at least 40 wt. %. In the same or still further embodiments, this combined total is from at least 50 wt. %. In the same or yet other embodiments, this combined total is from at least 60 wt. %. In the same or still other embodiments, this combined total is from at least 75 wt. %.

The weight ratio of the at least one antimicrobial surfactant to the at least one solvent can range from about 3:1 to about 1:2. In some embodiments, the weight ratio ranges from about 2:1 to about 1:2. In some embodiments, the weight ratio ranges from about 2:1 to about 1:1.

The concentrated solution can further comprise an effective amount of at least one additional antimicrobial agent. The at least one additional antimicrobial agent can be selected from the group consisting of highly soluble antimicrobial carboxylic acids and salts thereof. In some embodiments, the highly soluble antimicrobial carboxylic acids and salts thereof are selected from the group consisting of acetic acid, citric acid, dimethylol propionic acid, formic acid, glutaric acid, glycolic acid, mandelic acid, lactic acid, maleic acid, malic acid, malonic acid, oxalic acid, propionic acid, succinic acid, sulfosuccinic acid, and salts thereof. In the same or other embodiments, the highly soluble antimicrobial carboxylic acids and salts thereof are selected from the group consisting of citric acid, lactic acid, glycolic acid, mandelic acid, malic acid, oxalic acid, and salts thereof. When used, the at least one additional antimicrobial agent can be present in a concentration of from about 3 wt. % to about 30 wt. %.

Depending on the desired end use, the concentrated solution can further comprise at least one optional functional ingredient in an effective amount. The functional ingredient can be selected from the group consisting of abrasive agents, additional solvents, additional surfactants, allergicides, anti-foaming agents, antioxidants, anti-pilling agents, anti-redeposition agents, anti-static agents, anti-wrinkling agents, buffering agents, builders, brightening agents, chelating agents, color protection agents, corrosion inhibitors, dyes, dye transfer inhibition agents, emulsifiers, enzymes, film forming agents, flame retardants, foaming agents, fragrances, hydrotropes, lubricants, metal salts, mildew removing agents, odor removal agents, odor capturing agents, peracid precursors, pH adjusting agents, plasticizers, preservatives, radical scavengers, rheology modifiers, skin conditioning agents, softening agents, soil releasing agents, soil shielding agents, soil suspenders, souring agents, stabilizing agents, ultraviolet light protection agents, vitamins, water repellency agents, and wound healing agents.

In some embodiments, an effective amount of at least one acid pH adjusting agent is present. In some embodiments, the at least one acid pH adjusting agent has a pKa of less than 2.5 and is selected from the group consisting of methane sulfonic acid, toluene sulfonic acid, benzenesulfonic acid, sulfosalicylic acid, naphthalene disulfonic acid, sulfosuccinic acid, sulfuric acid, phosphoric acid, and sulfamic acid.

In some embodiments, water is present, and the pH of the solution is from about-3 to about 1. In some embodiments, the pH of the solution is from about-2 to about 0.5.

In some embodiments, an effective amount of at least one corrosion inhibitor is present.

An effective ready-to-use (RTU) solution according to a second aspect of the invention can be prepared by mixing the concentrated solution with an aqueous diluent at a ratio (solution:diluent) of up to about 1:1024, about 1:512, about 1:256, about 1:128, or about 1:64. The RTU solution will have a pH from about 1 to about 6, and preferably from about 1 to about 5. Depending on the embodiment and dilution ratio, the RTU solution will pass the Germicidal Spray Testing AOAC 961.02 test method at room temperature and a contact time of no greater than 10, 7, 5, or 3 minutes against S. aureus.

According to a third aspect, the invention provides a kit of parts wherein the parts are packaged separately in at least two compartments and, wherein, when the parts are combined, a solution according to the first aspect or second is formed. A fourth aspect of the invention provides a method of reducing a microbial load on a surface contaminated with microbes, the method comprising:
d. identifying a surface contaminated with microbes;
e. providing a concentrated solution according to any one of claims 1 to 41;
f. mixing the solution with an aqueous diluent at a ratio of from about 1:64 to about 1:1024 (solution:diluent) to produce a ready-to-use (RTU) solution;
g. applying an effective amount of the RTU solution to the surface for a contact of up to 10 minutes to reduce the microbial load on the surface.

In some embodiments, the surface is a hard surface, and the method is not a method of medical treatment. However, the method can also be used to reduce the number of microbes on skin or plants to treat or prevent a disease or condition.

Any method of applying an antimicrobial solution to a surface in need of microbial reduction can be used such as by spraying, wiping, immersing, fogging, etc. Solutions according to the invention can be applied in diluted or concentrated form. They can also be impregnated into a wipe material that can be used on surfaces, with or without prior dipping in water or an aqueous diluent.

A fifth aspect of the invention provides a method of making a concentrated antimicrobial solution containing at least one sparingly soluble antimicrobial agent in a concentration that exceeds its solubility in water by 20 to 60 times, the method comprising dissolving 20 to 60 times the amount of said at least one sparingly soluble antimicrobial agent than the amount that would dissolve in water in a solution comprising:
(a) an effective amount of at least one antimicrobial surfactant selected from the group consisting of anionic surfactants and amphoteric surfactants; and
(b) an effective amount at least one solvent selected from the group consisting of esteramides, diamides, branched dibasic esters, and alkyl pyrrolidones;
wherein, the weight ratio of the at least one antimicrobial surfactant to the at least one solvent is from 1:3 to 3:1, and optionally adding at least one functional ingredient to form a final solution, wherein the final solution has a pH less than 0.6.

In certain embodiments, the solution is substantially free of one or more of cationic surfactants, triclocarban, preservatives (such as pyrithione and parabens), halogenic phenolic compounds (such as triclosan), and other antimicrobial agents such as aldehydes, quaternary ammonium compounds (“quats”), chlorine bleach, and peroxide compounds (e.g., hydrogen peroxide). Most, if not all, of these compounds have drawbacks. For example, cationic surfactants can be corrosive to biological membranes such as those present in the eyes and the gastrointestinal tract. Triclosan is known to be toxic to aquatic organisms. Triclocarban is believed to be carcinogenic and exposure to high doses may cause toxicity in the blood system, liver, kidney and testes. Certain alcoholic solvents such as ethanol and isopropyl alcohol at high concentrations can be irritating to skin, nasal and esophageal membranes, and may be flammable. The drawbacks of the recited antimicrobial agents are described above.

Embodiments of the invention can be used at extremely high dilution ratios, i.e., as high as 1:1024 (solution:diluent) or, in some cases, even higher. At the same time, the diluted solutions are low in toxicity, environmentally safe, non-irritating to the user, and low corrosive to environmental surfaces.

### DETAILED DESCRIPTION

Embodiments of the invention will now be described to facilitate a better understanding of the invention. For the sake of clarity, the following terms used in the description and claims are defined as follows.

The present solutions are “phase stable,” which means that the solution is clear and in a single phase both at the time of preparation and after at least one cycle of cooling to −20° C. for at least 12 hours and warming to room temperature. This process is referred to herein as “freezing and thawing” even though, some solutions may not freeze depending on the nature and amount of solvents employed. By “single phase” is meant that the solution is homogeneous and that no solids and permanently distinct liquid phases are present. Concentrated solutions according to the invention are intended to be diluted with a diluent prior to use. However, this does not preclude using these solutions undiluted or using lower dilution ratios to enhance the activity of the solution, e.g., to achieve a desired level of microbial reduction at shorter contact times. Embodiments of concentrated solutions according to the invention, when diluted with an aqueous diluent at a weight ratio of from about 1:64, about 1:128, or about 1:160, and up to about 1:256, about 1:320, about 1:512, about 1:600, about 1700, about 1:800, about 1:900, about 1:1000 or about 1:1024 (solution:diluent), will have a pH of from about 1 to about 6, and pass the Germicidal Spray Testing AOAC 961.02 test method (“GST method”), at room temperature, and a contact time of no greater than 10 minutes against Staphylococcus aureus (S. aureus). Ready-to-use (RTU) diluted solutions are also within the scope of the present invention.

References herein to the GST method are not intended to exclude compliance with other standardized test methods for microbial reduction known in the art (e.g., those established by the American Society for Testing and Materials (ASTM), Organisation for Economic Co-operation and Development (OECD), Association of Official Agricultural Chemists (AOAC), and European Standards (EN)).

“Antimicrobial surfactant” means a surfactant that has antimicrobial properties. These are known to the person skilled in the art. Examples of antimicrobial surfactants are described herein.

“Acid pH adjusting agent” means any compound or ingredient that functions to reduce the pH of an aqueous solution. In some embodiments, the acid pH adjusting agent has a pKa of less than 4, 3, 2.3, or 2.

The skilled person will appreciate that, in some cases, the same compound or ingredient can perform multiple functions and/or belong to multiple classes of compounds. For example, some antimicrobial surfactants, such as dodecyl benzene sulphonic acid, can also function as an acid pH adjusting agent. Salicylic acid is a sparingly soluble antimicrobial agent and can also function as an acid pH adjusting agent and chelating agent. Furthermore, salicylic acid is both a phenolic compound and a C5-C28 carboxylic acid, which classes are described herein. Some acid pH adjusting agents, such as phosphoric acid, can also possess pH buffering capabilities.

The term “comprising” means “including without limitation.” Thus, a solution comprising a list of ingredients or compounds may include additional ingredients or compounds not expressly recited. The term “consisting of” (in the context of a solution) means “including the listed ingredients or compounds and such additional ingredients or compounds that can be present as natural or commercial impurities or additives.” Natural and commercial impurities and additives will be apparent to the person of ordinary skill in the art with reference to the literature provided by manufacturers of the ingredients used in the present solutions. This literature includes product specification sheets and certificates of analysis (C of A). The term “consisting essentially of” means “consisting of” the listed ingredients (as defined herein) and additional ingredients that would not materially affect the basic and novel properties of the solution. The “basic and novel properties” of the present concentrated solution are that the solutions are “phase stable” and form antimicrobially-effective RTU solutions following dilution at a ratio (solution:diluent) of from about 1:64 to about 1:1024.

The singular forms “a”, “an”, and “the” include the plural forms unless the content or context clearly dictates otherwise. Thus, for example, reference to “an antimicrobial agent” includes reference to two or more antimicrobial agents, unless the content or context clearly dictates otherwise.

When used herein, the term “effective amount” vis-à-vis an ingredient or compound means that amount that would bring about the desired effect, based on the purpose and function of the ingredient or compound, or solution in which it is used. What constitutes an effective amount will be readily determinable by the person of ordinary skill in the art having regard to the teachings herein and information in the public domain. For example, an effective amount of a pH adjusting agent is that amount which would cause the pH of the solution to reach a desired value. An “effective amount” of an antimicrobial agent means an amount that, together with other ingredients in the solution, will cause the solution to achieve the desired level of antimicrobial efficacy based on the intended application.

As used herein, “wt. %”, “% w/w,” “weight percent,” and variations thereof refer to the weight of an ingredient or compound divided by the total weight of the solution containing that ingredient or compound, multiplied by 100. It is understood that the total weight percent of all ingredients and/or compounds in a solution will not exceed 100 wt. %.

As used herein, the term “about” refers to a variation in a specified numerical value that can occur, for example, due to typical measuring and liquid handling procedures used for making solutions under real world conditions, differences in the manufacture, source, or purity of the ingredients used to make the solutions, etc. The term “about” also encompasses variations resulting from different equilibrium conditions for a solution resulting from a particular initial mixture. For the sake of clarity, the term “about” includes variations in the expressed value of +5%. Whether or not a value is modified by the term “about,” the claims include equivalents to the values.

“Room temperature” means a temperature from 18° C. to 25° C., inclusive.

As used herein, the term “q.s.” means “quantum sufficit” or “quantum satis,” which are Latin terms meaning “the amount which is enough,” or “as much as is sufficient.”

The expression, “substantially free of X,” means that “X” is not deliberately added but may be present as an impurity or due to other factors. For the sake of clarity, a solution that is “substantially free of X” will not contain X or contain only up to 0.01, 0.02, 0.03, 0.04, 0.05, 0.06, 0.07, 0.08, 0.09, 0.1, 0.2, 0.3, 0.4, or 0.5 wt. % X.

As used herein, the term “alkyl” or “alkyl groups” refers to saturated hydrocarbons having one or more carbon atoms, including straight-chain alkyl groups (e.g., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (e.g., cyclopropyl, cyclopentyl, cyclohexyl, cycloheptyl, cyclooctyl, etc.), branched-chain alkyl groups (e.g., isopropyl, tert-butyl, sec-butyl, isobutyl, etc.), and alkyl-substituted alkyl groups (e.g., alkyl-substituted cycloalkyl groups and cycloalkyl-substituted alkyl groups).

Unless otherwise specified, the term “alkyl” includes both “unsubstituted alkyls” and “substituted alkyls.” As used herein, the term “substituted alkyls” refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonate, phosphine, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonates, sulfamoyl, sulfonamido, nitro, trifluoromethyl, cyano, azido, heterocyclic, alkylaryl, or aromatic (including heteroaromatic) groups.

In some embodiments, substituted alkyls can include a heterocyclic group. As used herein, the term “heterocyclic group” includes closed ring structures analogous to carbocyclic groups in which one or more of the carbon atoms in the ring is an element other than carbon, for example, nitrogen, sulfur or oxygen. Heterocyclic groups can be saturated or unsaturated. Exemplary heterocyclic groups include, but are not limited to, aziridine, ethylene oxide (epoxides, oxiranes), thiirane (episulfides), dioxirane, azetidine, oxetane, thietane, dioxetane, dithietane, dithiete, azolidine, pyrrolidine, pyrroline, oxolane, dihydrofuran, and furan.

The ranges of values recited herein are intended to include all values within the ranges. Thus, for example, a range of 0.01 to 4.5 wt. % is intended to include values such as from 0.02, 0.03, or 0.04, etc. wt. % and up to 4.4, 4.3, or 4.2, etc. wt. %.

The present specification contemplates the possibility of omitting any components listed herein whether they are expressly named as included or excluded from solutions according to the invention.

The present specification also expressly contemplates using the compounds recited herein in different isomeric forms, to the extent that they exist in different isomeric forms and can be expected to work.

The term “microbial load” means the number of microorganisms present on a surface.

As used herein, the term “microorganism” refers to any non-cellular or unicellular (including colonial) organism. Microorganisms include bacterial spores (e.g., B. subtilis), mycobacteria, protozoa, fungal spores, vegetative fungi, yeast, vegetative bacteria (including cyanobacteria), enveloped and non-enveloped viruses, and other viruses (e.g., virinos, viroids, phages). The term is used interchangeably herein with “microbe.”

As used herein, the term “sanitizer” refers to an agent or composition that reduces the number of vegetative bacteria by at least a 99.9% (i.e. at least a 3 log₁₀ order reduction) using standardized test methods, such as the method set out in Germicidal and Detergent Sanitizing Action of Disinfectants, Official Methods of Analysis of the Association of Official Analytical Chemists, paragraph 960.09 and applicable sections, 15th Edition, 1990 (EPA Guideline 91-2).

As used herein, the term “disinfectant” refers to an agent that kills most microorganisms, including most recognized pathogenic microorganisms, providing at least a 99.999% reduction of bacteria, and at least a 99.9% reduction of viruses. The testing could be conducted using the procedure described in A.O.A.C. Use Dilution Methods, Official Methods of Analysis of the Association of Official Analytical Chemists, paragraph 955.14 and applicable sections, 15th Edition, 1990 (EPA Guideline 91-2), Germicidal Spray Test method for sanitizers and disinfectants (described further below), or any other methods mandated by various regulatory agencies that have not been specifically outlined herein. As used herein, the term “high level disinfectant” refers to a composition that kills substantially all organisms, except high levels of bacterial spores, and is a chemical germicide cleared for marketing as a high-level disinfectant or sterilant by the Food and Drug Administration in United States.

Sterilants, also referred to as chemical sterilants and chemosterilants, are chemical agents that can be used in the sterilization of articles. Sterilization involves very high levels of microbial kill including killing hardy microbes such as bacterial spores, and, in certain cases, a complete kill of all microbes.

As used in this invention, the term “sporicide” refers to a physical or chemical agent or process having the ability to kill at least 50% of the number of spore cells, such as Bacillus cereus or Bacillus subtilis (B. subtilis), Clostridioides difficile (C. difficile, formerly called Clostridium difficile), Bacillus atrophaeus, Chaetomium globosum and Paenibacillus chibensis. Embodiments of the invention can be formulated to have sporicidal activity when used at lower dilution ratios.

### Sparingly Soluble Antimicrobial Agent

Solutions according to the invention employ an effective amount of at least one sparingly soluble antimicrobial agent which can be a single compound or ingredient comprising a plurality of compounds. “Sparingly soluble” means that the antimicrobial agent has a water solubility of from about 0.005 wt. % to about 3 wt. % at room temperature and pH below 7. Only compounds known to the skilled person to have antimicrobial properties are intended to be within the scope of the invention.

The sparingly soluble antimicrobial agents according to the invention can be classified as antimicrobial phenolic compounds, C5-C28 carboxylic acids and salts thereof, terpenes, terpenoids, and essential oils (which may contain terpenes and/or terpenoids). In some cases, a compound (e.g., salicylic acid) can fall into more than one of these categories.

The phenolic compounds useful herein can be “non-halogenic phenolic compounds,” meaning antimicrobial compounds that incorporate a phenol functional group and which do not contain a halogen compound, such as, fluorine, chlorine, bromine, and iodine. Many of these compounds are undesirable because of their unfavourable environmental and toxicity profiles. Examples of non-halogenic phenolic compounds that can be used include, without limitation, methyl phenol; dimethyl phenol; trimethyl phenol; diphenol; polyphenols; benzyloxy phenol; menthol; phenylphenol; hydroxyacetophenone; hydroxypropiophenone; hydroxyalkyl phenol; gingerol; naphthol, naphthalenediol; substituted or unsubstituted cyclic hydroxy carboxylic acids containing at least one hydroxyl group on the ring such as salicylic acid, capryloyl salicylic acid, methylsalicylic acid, coumaric acid, ferulic acid, diferulic acid, sinapic acid, caffeic acid, protocatechuic acid, syringic acid, vanillic acid, gallic acid, anthranilic acid; and ellagic acid. In some embodiments, the sparingly soluble antimicrobial agent is selected from the group consisting of salicylic acid, hydroxyacetophenone, benzyloxy phenol, phenylphenol, methylsalicylic acid, gallic acid, anthranilic acid, and menthol.

The C5-C28 carboxylic acids and salts thereof can be saturated or unsaturated, substituted or unsubstituted, cyclic or acyclic, and branched or unbranched. Examples C5-C28 carboxylic acids and salt thereof that can be used include, without limitation, pivalic acid, hexanoic acid, isohexanoic acid, hexenoic acid, hydroxyhexanoic acid, heptanoic acid, isoheptanoic acid, hepteneoic acid, hydroxyheptanoic acid, octanoic acid, isooctanoic acid, octanoic acid, hydroxyoctanoic acid, decanoic acid, isodecanoic acid, hydroxydecanoic acid, dodecanoic acid, dodecenoic acid, isodecanoic acid, lauroyl-1-lactylic acid, lauroyl-2-lactylic acid, myristoyl-1-lactylic acid, myristoyl-2-lactylic acid, palmitoyl-1-lactylic acid, palmitoyl-2-lactylic acid, steroyl-2-lactylic acid, cocoyl sarcosine, myristoyl sarcosine, oleoyl sarcosine, C10-C28 alkyl ether carboxylic acids, benzoic acid, phenoxyacetic acid, phenylacetic acid, salicylic acid, hydroxybenzoic acid, dihydroxybenzoic acid, tropic acid, phthalic acid, terephthalic acid, methylbenzoic acid, anthranilic acid, furoic acid, cinnamic acid, hydroxycinnamic acid, picolinic acid, dipicolinic acid, pyridinedicarboxylic acid, pyridinecarboxylic acid, and hydratropic acid, and salts thereof. In some embodiments, the C5-C28 carboxylic acids are selected from the group consisting of hexanoic acid, heptanoic acid, octanoic acid, nonanoic acid, decanoic acid, benzoic acid, phenoxyacetic acid, phenylacetic acid, salicylic acid, phthalic acid, furoic acid, cinnamic acid, and salts thereof.

Examples of terpenes and terpenoids include, without limitation, thymol, carvacrol, eugenol, linalool, linalyl acetate, citronellol, geraniol, cineol/eucalyptol, thujone, pinene, neral, borneol, terpenyl acetate, limonene, terpinene, methyl eugenol, acetyl eugenol, myrcene, terpinolene, terpineol, anisol, citronellal, neem oil and its extracts, tea tree oil and its extracts, neryl acetate, methyl cinnamate, geranyl acetate, and bergamottin. In some embodiments, the essential oils, terpenes and terpenoids are selected from the group consisting of thymol, carvacrol, eugenol, limonene, cinnamaldehyde, pinene, terpinene, terpinol, neem oil, tea tree oil, eucalyptus oil, eucalyptol, and anisole.

### Antimicrobial Surfactant

The present solutions contain an effective amount of at least one antimicrobial surfactant (different from the sparingly soluble antimicrobial agent) selected from the group consisting of anionic surfactants and amphoteric surfactants. As used herein, the term “anionic” means containing at least one dissociable acidic moiety or salt thereof that can lead to a negative charge upon dissociation. Such moieties could be based on acids that contain sulfur, phosphorous, or carboxyl at their core compartment, resulting in sulfonates, sulfates, phosphates, phosphonates, and carboxylates upon dissociation. As used herein, the term “amphoteric” means either molecules that contain both acidic and basic sites such as, for example, an amino acid containing both amino (basic) and acid (e.g., carboxylic acid, acidic) functional groups; or Zwitterion molecules which possess both positive and negative charges within the same molecule. The charges of the latter may be either dependent on or independent of the pH of the composition.

The anionic and amphoteric surfactants must be antimicrobial, meaning that they must function to kill microbes such as positive and gram-negative bacteria, and/or enveloped viruses. Antimicrobial anionic and amphoteric surfactants are known in the art. Exemplary antimicrobial anionic surfactants include C8-C28 sulfonates, C8-C28 sulfates, C8-C28 ether sulfates, C8-C28 ether sulfonates, C8-C28 olefin sulfonates, C8-C28 olefin sulfates, C6-C28 carboxylates, C6-C28 ether carboxylates, C8-C28 sulfonated carboxylates, C8-C28 phosphates, C8-C28 phosphonates, C8-C28 phosphate esters, C8-C28 taurates, acid forms of the aforementioned classes, and C8-C24 amino acid based surfactants. Antimicrobial amphoteric surfactants include alkyl betaines, e.g., lauryl betaine; alkylamido betaines, e.g., cocamidopropyl betaine and cocohexadecyl dimethylbetaine; alkylamido sultaines, e.g., cocamidopropyl hydroxysultaine; (mono- and di-) amphocarboxylates, e.g., sodium cocoamphoacetate, sodium lauroamphoacetate, sodium capryloamphoacetate, disodium cocoamphodiacetate, disodium lauroamphodiacetate, disodium capryloamphodiacetate, disodium cocoamphodipropionate, disodium lauroamphodipropionate, disodium caprylamphodipropionate, disodium capryloamphodipropionate, and C8-C22 alkyl amine oxides, e.g., octyldimethylamine oxide, decyldimethylamine oxide, dodecyldimethylamine oxide, iso-dodecyldimethyl amine oxide, myristyldimethylamine oxide, myristyl/cetyldimethylamine oxide, and cocodimethylamine oxide.

In some embodiments, the antimicrobial amphoteric surfactants are selected from the group consisting of C8-C24 alkyl amine oxides, alkyl betaines, alkylamido sultaines, and amphocarboxylates.

The antimicrobial surfactants according to the invention have multiple functions—they help stabilize both the concentrate and RTU versions of the solution (i.e., provide a clear, single-phase solution), help with soil penetration and cleaning, and enhance the antimicrobial activity of the solution so that effective solutions can be achieved even at high dilution ratios. Also, since the RTU solutions must have water, the antimicrobial surfactants are those surfactants that are soluble in water, i.e., have a solubility in water of at least 0.01% at room temperature.

The selection of the sparingly soluble antimicrobial agent and antimicrobial surfactant, and their amounts used, will provide a solution that, when diluted with an aqueous diluent at a ratio (solution:diluent) of from about 1:64 and up to about 1:1024, about 1:512, about 1:256, or about 1:128. will have a pH of from about 1 to about 6 and pass the Germicidal Spray Testing AOAC 961.02 test method, at room temperature, and a contact time of no greater than 10 minutes against S. aureus.

### Solvent

The present solutions employ an effective amount of at least one solvent selected from the group consisting of esteramides, diamides, alkyl pyrrolidones, and branched dibasic esters. The solvents are used in combination with the antimicrobial surfactant to solubilize the sparingly soluble antimicrobial agent in unexpectedly high amounts and render the end solution phase stable. Some solvents, e.g., N-butyl pyrrolidone (see, e.g., U.S. Pat. No. 11,129,385 to Alderson, incorporated herein by reference) also possess antimicrobial properties and therefore may contribute to microbicidal efficacy depending on the end use concentration and target organisms.

The esteramides that are used in solutions according to the present invention are according to Formula I below:

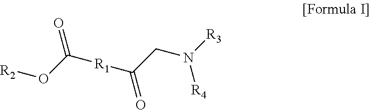

wherein R₁ is a C2-C8 branched or linear alkyl group;
R₂ is a C1-C6 linear, branched or cyclic alkyl group; and
R₃ and R₄ are independently methyl or ethyl.

In some embodiments, R₃ and R₄ are the same.

The diamides that are used in solutions according to the present invention are according to Formula II below:

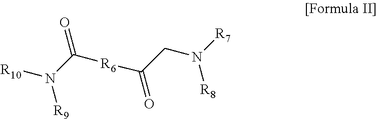

wherein R₆ is a C2-C8 branched or linear alkyl group; and
R₇, R₈, R₉ and R₁₀ are independently methyl or ethyl.

In some embodiments, R₇ and R₈ are the same. In the same or other embodiments, R₉ and R₁₀ are the same. For example, all of R₇, R₈, R₉ and R₁₀ can be the same in some embodiments.

An exemplary solvent is sold in association with the trademark Polarclean™, which is a mixture of the following esteramide and diamide in a ratio of 20:1 (esteramide:diamide) and has a purity of >98%—

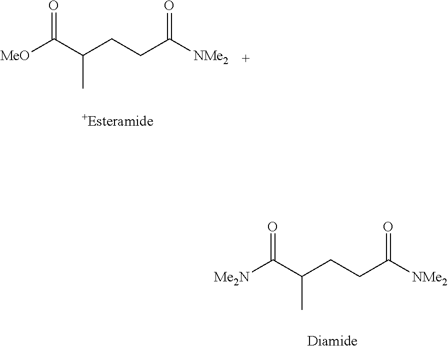

Alkyl pyrrolidones that can be used as solvents in embodiments of the present invention are those according to the following Formula III:

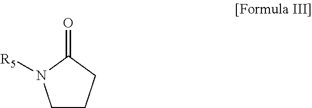

wherein R₅ is a branched or unbranched, saturated or unsaturated, substituted or unsubstituted C2 to C6 alkyl chain. In some embodiments, the alkyl chain is a C3 to C5 alkyl chain. In the same or other embodiments, R₅ is a C3 alkyl chain substituted with a methoxy group. In certain embodiments, the compound of Formula III is a C4 N-alkyl-gamma-butyrolactam. In certain embodiments, the C4 N-alkyl-gamma-butyrolactam is selected from the group consisting of N-butyl-gamma-butyrolactam, N-isobutyl-gamma-butyrolactam, N-methoxypropyl-gamma-butyrolactam, and combinations thereof. In further embodiments, the C4 N-alkyl-gamma-butyrolactam is N-butyl-gamma-butyrolactam (also referred to as N-butyl pyrrolidone) which is a solvent sold in association with the trademark Tamisolve™ NxG by Eastman.

The branched dibasic esters that are used in solutions according to the invention are according to Formula IV below:

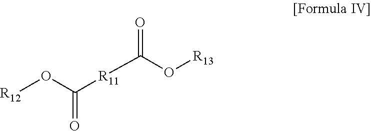

wherein R₁₁ is a C2-C8 branched alkyl group; and
R₁₂ and R₁₃ are independently a C1-C4 linear or branched alkyl group.

In some embodiments, R₁₂ and R₁₃ are the same.

An example solvent that contains a branched dibasic ester is a solvent sold in association with the trademark Rhodiasolv® IRIS which contains dimethyl-2-methyl glutarate (Cas Number 14035-94-0). This compound has the following formula:

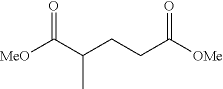

The present antimicrobial surfactant and solvent collectively function to solubilize the at least one sparingly soluble antimicrobial agent to provide a phase stable concentrated solution.

### Functional Ingredients

Depending on the desired properties of the solution, an effective amount of one or more optional additional functional ingredients can be included such as abrasive agents, additional antimicrobial agents, additional solvents, additional surfactants, allergicides, anti-foaming agents, antioxidants, anti-pilling agents, anti-redeposition agents, anti-static agents, anti-wrinkling agents, buffering agents, builders, brightening agents, chelating agents, color protection agents, corrosion inhibitors, dyes, dye transfer inhibition agents, emulsifiers, enzymes (such as proteases and lipases), film forming agents, flame retardants, foaming agents, fragrances, hydrotropes (e.g. sodium xylene sulfonate), lubricants, mildew removing agents, odor removal agents, odor capturing agents, peracid precursors, pH adjusting agents, plasticizers, preservatives, radical scavengers, rheology modifiers, skin conditioning agents, softening agents, soil releasing agents, soil shielding agents, soil suspenders, souring agents, stabilizing agents, ultraviolet light protection agents, vitamins, water repellency agents, and wound healing agents.

In some embodiments, the concentrated solution will have at least one functional ingredient selected from the group consisting of additional antimicrobial agents, additional solvents, additional surfactants, anti-foaming agents, anti-redeposition agents, buffering agents, builders, chelating agents, corrosion inhibitors, dyes, emulsifiers, foaming agents, fragrances, hydrotropes (e.g. sodium xylene sulfonate), peracid precursors, pH adjusting agents, rheology modifiers, soil releasing agents, and stabilizing agents.

Additional antimicrobial agents include all known antimicrobial agents having a solubility greater than 3 wt. % (herein referred to as “highly soluble antimicrobial agents”). Examples include certain carboxylic acids and salts thereof, C1-C10 alcohols, aldehydes, halogenated phenolic compounds, peroxygen compounds, and metal salts. When used, they would be present in amounts that do not give rise to undesirable properties, such as toxicity to the user and environment, corrosiveness to surfaces, unsafe handling, and so forth.

The highly soluble carboxylic acids and salts thereof include, without limitation, acetic acid, citric acid, dimethylol propionic acid, formic acid, mandelic acid, glutaric acid, glycolic acid, lactic acid, maleic acid, malic acid, malonic acid, oxalic acid, propionic acid, succinic acid, sulfosuccinic acid, and salts thereof. In some embodiments, the carboxylic acids and salts thereof are selected from the group consisting of citric acid, lactic acid, glycolic acid, mandelic acid, glutaric acid, malic acid, oxalic acid and salts thereof. Highly soluble antimicrobial carboxylic acids and salts thereof can be present in a concentration of from about 1 wt. % to about 40 wt. %.

In some embodiments, the peroxygen compounds can be selected from the group consisting of hydrogen peroxide, hydrogen peroxide releasing agents, and peroxycarboxylic acids and salts thereof.

Additional surfactants include nonionic surfactants, non-antimicrobial anionic surfactants, non-antimicrobial amphoteric surfactants, and those cationic surfactants that are compatible with the other ingredients and compounds in the present solutions.

Exemplary nonionic surfactants that can be used include, without limitation, alkoxylates made from ethylene oxide (EO), propylene oxide (PO), and butylene oxide (BO). Suitable alkoxylated surfactants include homo or copolymers or terpolymers, capped EO/PO/BO copolymers, alcohol alkoxylates, capped alcohol alkoxylates, mixtures thereof, or the like. Suitable alkoxylated surfactants which also function as solvents include EO/PO block copolymers, such as the Pluronic® and reverse Pluronic surfactants; alcohol alkoxylates such as Dehypon® LS-54, and Dehypon® LS-36 capped alcohol alkoxylates, such as Plurafac® LF221 and Tegotens® EC11. More specifically, the composition of the present specification can include an alkoxylated primary or secondary alcohol having from 8 to 18 carbon atoms reacted with from 2 to 12 moles of ethylene, and/or propylene, and/or butylene oxide. In an embodiment, the nonionic surfactant has from 3 to 18 moles of alkylene oxide, in another embodiment from 3 to about 10 moles of ethylene oxide (EO), and in yet another embodiment about 7 moles of EO. Examples include lauryl alcohol ethoxylated with 3 moles of ethylene oxide (EO), coco alcohol ethoxylated with 3 moles EO, stearyl alcohol ethoxylated with 5 moles EO, mixed C₁₂-C₁₅ alcohol ethoxylated with 7 moles EO, mixed secondary C₁₁-C₁₅ alcohol ethoxylated with 7 moles EO, mixed C₉-C₁₁ linear alcohol ethoxylated with 6 moles EO and the like. In some embodiments, the nonionic surfactant can have from 8 to 15 carbon atoms in the alkyl group. In an embodiment, the composition comprises the alcohol alkoxylates, particularly the alcohol ethoxylates and propoxylates, especially the mixed ethoxylates and propoxylates, particularly with 3-7 oxyethylene (EO) units and 3-7 oxypropylene (PO) units such as the alcohol Dehypon™ available from Cognis Corporation, having 5 EO units and 4 PO units.

The semi-polar type of nonionic surface-active agents are another class of nonionic surfactant useful in solutions according to the invention. Semi-polar nonionic surfactants include phosphine oxides, sulfoxides and their alkoxylated derivatives.

pH adjusting agents that can be used include acid and basic pH adjusting agents. Examples of pH adjusting agents include, without limitation, inorganic acids (e.g., phosphoric acid) and salts thereof, organic acids (e.g., citric acid, methane sulfonic acid, p-toluene sulfonic acid) and salts thereof, and alkaline agents (e.g., potassium hydroxide and sodium hydroxide). In some embodiments, the acid pH adjusting agents include those having a pKa of less than 2.5, such as p-toluene sulfonic acid, methane sulfonic acid, benzenesulfonic acid, naphthalene disulfonic acid, sulfosalicylic acid, sulfuric acid, sulfosuccinic acid, sulfamic acid, phosphoric acid, and sodium bisulfate.

Examples of basic pH adjusting agents include potassium hydroxide, sodium hydroxide, lithium hydroxide, triethanolamine, monoethanolamine, calcium hydroxide, sodium carbonate, sodium bicarbonate, barium carbonate, tetramethylammonium hydroxide, guanidine, ammonium hydroxide, ammonia, and sodium amide.

Buffering agents may also be used including mixtures of acids and their conjugate bases, or acidic molecules containing more than one acidic functional group. The pH adjusting agents and buffering agents are selected and used in an amount such that the final pH of a ready-to-use diluted solution is in the range of from about 1, 1.5, 2, or 2.5 to about 6, 5.5, 5, 4.5, 4, 3.5, or 3. For concentrated solutions, the pH can range from about-3 to about 1, or from about-2 to about 0 if some amount of water is present in order for pH to be measurable.

Chelating agents that can be included include, without limitation, 1-hydroxyethane-1,1-diphosphonic acid (HEDP; also referred to as etidronic acid), ethylenediaminetetraacetic acid (EDTA), glutamic acid diacetic acid (GLDA), methylglycine diacetic acid (MGDA), polymandelic acid, diethylenetriaminepentaacetic acid (DTPA), N-(hydroxyethyl)-ethylenediaminetriacetic acid (HEDTA), nitrilotriacetic acid (NTA), 2-hydroxyethyliminodiacetic acid (HEIDA), benzoic acid, aminobenzoic acid, citric acid, iminodisuccinic acid, polyaspartic acid, phosphoric acid, tripolyphosphate, amino tri(methylene phosphonic acid) (ATMP), diethylenetriaminepenta(methylene phosphonic acid), 2-hydroxy ethylimino bis(methylene phosphonic acid), ethylene diamine tetra(methylene phosphonic acid), hexamethylenediamine-tetra(methylene phosphonic) acid, and salts thereof. Exemplary concentrations of chelating agents used for the purpose of chelating metal ions can be from about 0.1, 0.5, 1, 2, 3, 4, or 5 wt. %, and up to about 15, 14, 13, 12, 11, 10, 9, 8, 7, or 6 wt. %.

An effective amount of a corrosion inhibitor (e.g., benzotriazole, tolyltriazole, molybdate salts, zinc dithiophosphate) can be used. Exemplary concentrations of corrosion inhibitors are from about 0.1, 0.5, 1, 1.5, 2, 2.5, 3, 3.5, 4, or 4.5 wt. % and up to about 10, 9, 8, 7, 6, 5.5, or 5 wt. %.

### General Comments

The sparingly soluble antimicrobial agent, antimicrobial surfactant, and the solvent are present in an “effective amount.” What constitutes an effective amount will depend on the identity of the agent, surfactant and solvent, and the identity of the optional ingredients that are also used. Concentrated solutions according to the invention will have a pH of up to about 1 and RTU versions will have a pH of from about 1 to about 6. pH adjusting agents can be used to achieve these pH values. The selection of ingredients or compounds and amounts used will provide a concentrated solution that is phase stable and that, when diluted with an aqueous diluent at a ratio of from about 1:64 and up to about 1:128, 1:256, 1:512, or 1:1024 (solution:diluent), or up to values below 1:1024 and between the other recited values, will have a pH of from about 1 to about 6, and pass the Germicidal Spray Testing AOAC 961.02 test method, at room temperature, and a contact time of no greater than 10 minutes against S. aureus. As mentioned above, some embodiments of the invention can satisfy these criteria at even higher dilution ratios.

The person skilled person in the art would appreciate what amounts of each compound and ingredient used in the present concentrated solutions would be an “effective amount” for a given application, based on the present teachings and information in the public domain. The combined total concentration of the antimicrobial surfactant(s) and the solvent(s) can be from about 30, 35, 40, 45, 50, 55, 60, 65, 70, 75, 80, 85, or 90 wt. %. Furthermore, the weight ratio of the at least one antimicrobial surfactant to the at least one solvent can be from about 3:1 to about 1:2, or from about 2:1 to about 1:2, or from about 2:1 to about 1:1.

The solubility of the at least one sparingly soluble antimicrobial agent can be at least 20, 25, 30, 35, 40, 45, 50, 55, 60, 65, 70, 75, 80, 85, 90, 95, or 100 times higher in concentrated solutions according to the invention than in water.

Various embodiments of the invention can have various levels of antimicrobial efficacy. For example, some embodiments, when diluted with an aqueous diluent at a ratio of from about 1:256 (solution:diluent), will provide an RTU solution having a pH of from about 1 to about 6, and that will pass the Germicidal Spray Testing AOAC 961.02 test method against S. aureus, at room temperature and a contact time of no greater than 10 minutes. The same or other embodiments, when diluted with an aqueous diluent at a ratio of from about 1:64 or 1:128 (solution:diluent), will provide an RTU solution having a pH of from about 1 to about 6, and that will pass the Germicidal Spray Testing AOAC 961.02 test method against S. aureus, at room temperature and a contact time of no greater than 5 minutes. Certain embodiments, when diluted with an aqueous diluent at a ratio of from about 1:256 (solution:diluent), will provide an RTU solution having a pH of from about 1 to about 6, and that will pass the Germicidal Spray Testing AOAC 961.02 test method against S. aureus, at room temperature and a contact time of no greater than 5 minutes. As well, some embodiments, when diluted with an aqueous diluent at a ratio of from about 1:64 or 1:128 (solution:diluent), will provide an RTU solution having a pH of from about 1 to about 6, and that will pass the Germicidal Spray Testing AOAC 961.02 test method against S. aureus, at room temperature and a contact time of no greater than 3 minutes.

The invention is further illustrated by the following non-limiting examples which employ the ingredients or compounds in TABLE A, wherein trademarks are identified as such using either the ® or the ™ logo. For the sake of convenience, the trademark indicia are used only in Table A and not elsewhere in the specification where the same term appears.

TABLE A

| INGREDIENT | PURITY | MFR* | COMPOUND | CATEGORY |
|---|---|---|---|---|
| Benzoic acid | 100% | Sigma-Aldrich | Benzoic acid | Acid/Sparingly |
| | | | | Soluble |
| | | | | Antimicrobial |
| | | | | Agent |
| Citric acid | 95-100% | Brenntag SE | Citric acid anhydrous | Acid/ |
| | | | | Antimicrobial |
| | | | | agent |
| Lactic acid | 80-90% | Sigma-Aldrich | Lactic acid | Acid/ |
| | | | | Antimicrobial |
| | | | | agent |
| Phenoxyacetic acid | 98% | Sigma-Aldrich | Phenoxyacetic acid | Acid/Sparingly |
| | | | | Soluble |
| | | | | Antimicrobial |
| | | | | Agent |
| Phenylacetic acid | 100% | Sigma-Aldrich | Phenylacetic acid | Acid/Sparingly |
| | | | | Soluble |
| | | | | Antimicrobial |
| | | | | Agent |
| Salicylic acid | >99% | Columbus | Salicylic acid | Acid/Sparingly |
| | | Chemical | | Soluble |
| | | Industries Inc. | | Antimicrobial |
| | | | | Agent |
| Lutropur ® MSA 100 | ≥99% | BASF Corporation | Methane sulfonic acid | Acid |
| p-Toluenesulfonic | 98% | Sigma-Aldrich | p-Toluenesulfonic acid | Acid |
| acid | | | monohydrate | |
| Dequest ® 2010 | 60% | Italmatch | Etidronic acid (1- | Chelating agent |
| | | Chemicals S.p.A. | Hydroxyethylidene-1,1- | |
| | | | diphosphonic acid or HEDP) | |
| Cobratec ® 35-G | 35% | CCC Chemicals | Benzotriazole | Corrosion |
| | | | | Inhibitor |
| Thymol | ≥98.5% | Sigma-Aldrich | Thymol | Essential Oil/ |
| | | | | Phenolic |
| | | | | Compound/ |
| | | | | Antimicrobial |
| | | | | Agent |
| Benzyl alcohol | 95-100% | Univar Inc. | Benzyl alcohol | Solvent/ |
| | | | | Antimicrobial |
| | | | | Agent |
| Butyl carbitol | >99% | Dow Chemical | Diethylene glycol monobutyl | Solvent |
| | | Company | ether | |
| Butyl lactate | 100% | Sigma-Aldrich | Butyl lactate | Solvent |
| Cyrene | 100% | Sigma-Aldrich | (1S,5R)-6,8- | Solvent |
| | | | dioxabicyclo[3.2.1]octan-4- | |
| | | | one | |
| Dimethyl adipate | 100% | Sigma-Aldrich | Dimethyl adipate | Solvent/ |
| | | | | Antimicrobial |
| | | | | Agent |
| Dimethyl succinate | 100% | Sigma-Aldrich | Dimethyl succinate | Solvent/ |
| | | | | Antimicrobial |
| | | | | Agent |
| Dowanol ® EPH | >99.5% | Dow Chemical | Phenoxyethanol | Solvent |
| | | Company | | |
| Dowanol ® TPM | 98% | Dow Chemical | Tripropyleneglycol methyl | Solvent |
| | | Company | ether | |
| Dowanol ® PnP | >99% | Dow Chemical | 1-Propoxy-2-propanol | Solvent |
| | | Company | | |
| Glycol ether DB | 99-100% | Univar Inc. | Butyl carbitol | Solvent |
| Glycol ether PM | 100% | Univar Inc. | 2-methoxy-1-methylethyl | Solvent |
| Acetate | | | acetate | |
| Methoxytriglycol | 90-100% | Dow Chemical | Triethylene glycol | Solvent |
| | | Company | monomethyl ether | |
| Mineral spirits 3139 | 95-100% | Brenntag Inc. | Solvent naphtha medium | Solvent |
| | | | aliphatic | |
| Propylene carbonate | 100% | Sigma-Aldrich | Propylene carbonate | Solvent |
| Propylene glycol | >99.8% | Univar Inc. | Propylene glycol | Solvent |
| Rhodiasolve ® Iris ™ | >85% | Solvay Inc. | Dimethyl 2-methylglutarate | Solvent |
| Rhodiasolve ® | 80-95% | Solvay Inc. | Pentanoic acid, 5- | Solvent |
| PolarClean ™ | | | (dimethylamino)-2-methyl-5- | |
| | | | oxo-, methyl ester + | |
| | | | Pentane-2-methyl-1,5-oxo- | |
| | | | 1,5-dimethylamine | |
| Tamisolve ™ NxG | >99.5% | Eastman | N-butyl pyrrolidone | Solvent/ |
| | | | | Antimicrobial |
| | | | | Agent |
| Akypo ® LF 4 | 90% | Kao Chemicals | Capryleth-9 carboxylic acid & | Anionic |
| | | | Hexeth-4 carboxylic acid | Surfactant/ |
| | | | | Antimicrobial |
| | | | | Agent |
| Bio-Soft ® S-101-LS | 90-100% | Stepan Chemical | Alkyl (C10-C16) | Anionic |
| | | Company | benzenesulfonic acid | Surfactant/ |
| | | Corporation | | Antimicrobial |
| | | | | Agent |
| Bioterge ® PAS-8S | 35-40% | Stepan Chemical | Sodium octane sulfonate | Anionic |
| | | Company | | Sulfonate |
| | | Corporation | | Surfactant/ |
| | | | | Antimicrobial |
| | | | | Agent |
| Calfoam ® ES-703 | 70-80% | Pilot Chemical | Alpha-sulfo-omega- | Anionic |
| | | | hydroxypoly (oxy-1,2- | Surfactant/ |
| | | | ethanidiyl) C10-16 alkyl | Antimicrobial |
| | | | ethers, | Agent |
| | | | Sodium salts | |
| Cocamidopropyl | 30% | LV Lomas | Cocamidopropyl betaine | Amphoteric |
| Betaine | | | | Surfactant/ |
| | | | | Antimicrobial |
| | | | | Agent |
| Dowfax ® C-6L | 40-50% | Dow Chemical | Disodium hexyl diphenyl | Anionic |
| | | Company | ether disulphonate | Surfactant/ |
| | | | | Antimicrobial |
| | | | | Agent |
| Dowfax ® C-10L | ≤46% | Dow Chemical | Benzenesulfonic acid, decyl | Anionic |
| | | Company | (sulfophenoxy)-, disodium | Surfactant/ |
| | | | salt & | Antimicrobial |
| | | | Benzenesulfonic acid, | Agent |
| | | | oxybis(decyl)-, disodium salt | |
| Hostapon ® CT Paste | 30% | Clariant | Sodium methyl cocoyl | Anionic |
| | | | taurate | Surfactant/ |
| | | | | Antimicrobial |
| | | | | Agent |
| Mackamine ® LO | 30% | Rhodia | Lauramine oxide | Amphoteric |
| | | | | Surfactant/ |
| | | | | Antimicrobial |
| | | | | Agent |
| Maprosyl ® 30-B | 30% | Stepan Chemical | Sodium lauroyl sarcosinate | Anionic |
| | | Company | | Surfactant/ |
| | | Corporation | | Antimicrobial |
| | | | | Agent |
| Multitrope ™ 1214- | 90-100% | Croda | Alkoxylated phosphate ester | Anionic |
| LQ-(MV) | | | | Surfactant/ |
| | | | | Antimicrobial |
| | | | | Agent |
| Texapon ® LS 30 | 28-30% | BASF Corporation | Sulfuric acid, mono-C10-16- | Anionic |
| | | | alkyl esters, sodium salts | Surfactant/ |
| | | | | Antimicrobial |
| | | | | Agent |
| Tomadol ® 91-6 | 100% | Air Products | C9-11 ethoxylated alcohols | Nonionic |
| | | | | Surfactant |
| Pluronic ® L62 | ≤100% | BASF Corporation | Polyoxypropylene- | Nonionic |
| | | | polyethylene block | Surfactant |
| | | | copolymer | |

*MFR = manufacturer

Solutions were prepared by mixing ingredients together in the order shown in the below tables. Low, medium, or high shear mechanical stirring methods were used as required to achieve a clear, single-phase solution at room temperature. p-Toluenesulfonic acid is an acidifying agent and was used to provide a concentrate solution that, when diluted with water by a ratio of solution:water of from about 1:64 and up to about 1:1024 would have a pH less than 6, preferably less than 5, and be antimicrobially-effective. p-Toluenesulfonic acid also provides hydrotroping functions. Citric acid was used for its functions as a chelating agent, buffering agent, and antimicrobial agent.

Certain solutions were subjected to a freeze thaw test described above after which visual assessments were conducted to identify any liquid phase separation or solid precipitation. Solutions that remained clear and in a single phase passed the freeze thaw test.

In the below tables, the amount of each compound or ingredient is expressed in terms of the wt. % of the raw material used. Since the raw materials, in some cases, have a concentration of the active(s) or compound(s) that is less than 100 wt. %, to determine the wt. % of the active(s) or compound(s) in the example solutions, one must multiple the purity (%) of the raw material specified in Table A with the wt. % of the raw material specified in the below tables, and divide by 100. For example, Solution 37 contains 35 wt. % AKYPO LF 4 which is a commercial solution containing capryleth-9 carboxylic acid and hexeth-4 carboxylic acid with a purity of 90%. Therefore, the combined total of capryleth-9 carboxylic acid and hexeth-4 carboxylic acid in Solution 37 is (90×35 wt. %)÷100=31.5 wt. %.

### Example 1—Solubility of Salicylic Acid in Different Solvent Solutions

Solutions 1-6 were prepared to evaluate the solubility of a sparingly soluble antimicrobial agent (i.e., salicylic acid) in a base solution. These solutions are summarized in Table 1 below. The solutions were visually inspected immediately following preparation and after storage at room temperature for 24 hours. Solution 2 was also subjected to the freeze thaw test described above and inspected after it was thawed. The results of these inspections are also summarized in Table 1 below.

TABLE 1

| | 1 | 2 | 3 | 4 | 5 | 6 |
|---|---|---|---|---|---|---|
| INGREDIENT | Wt. % | Wt. % | Wt. % | Wt. % | Wt. % | Wt. % |
| Tamisolve NxG | — | 18 | — | — | — | — |
| Dowanol PnP | — | — | 18 | — | — | — |
| Dowanol TPM | — | — | — | 18 | — | — |
| Dowanol EPH | — | — | — | — | 18 | — |
| Butyl Carbitol | — | — | — | — | — | 18 |
| Salicylic acid | 10 | 10 | 10 | 10 | 10 | 10 |
| Pluronic L62 | 5 | 5 | 5 | 5 | 5 | 5 |
| Dowfax C6L | 19 | 19 | 19 | 19 | 19 | 19 |
| (40% active) | | | | | | |
| Dowfax C10L | 4.3 | 4.3 | 4.3 | 4.3 | 4.3 | 4.3 |
| (40% active) | | | | | | |
| Citric acid | 12 | 12 | 12 | 12 | 12 | 12 |
| Cobratec 35-G | 1 | 1 | 1 | 1 | 1 | 1 |
| Dequest 2010 | 1.2 | 1.2 | 1.2 | 1.2 | 1.2 | 1.2 |
| Biosoft S-101 LS | 24.5 | 24.5 | 24.5 | 24.5 | 24.5 | 24.5 |
| Lutropur MSA 100 | 5 | 5 | 5 | 5 | 5 | 5 |
| pH | <0.6 | <0.6 | <0.6 | <0.6 | <0.6 | <0.6 |
| Clarity, upon | Cloudy | Clear, fully | Cloudy | Cloudy | Cloudy | Cloudy |
| prep: | | dissolved | | | | |
| Clarity, 24 hr | Cloudy | Clear, no | Precipitated | Precipitated | Cloudy | Cloudy |
| at room temp.: | | precipitates | | | | |
| Clarity, | — | Clear, no | — | — | — | — |
| freeze-thaw: | | precipitates | | | | |

Solution 1 contained no solvent. Solutions 2-6 contained solvents Tamisolve NxG, Dowanol PnP, Dowanol TPM, Dowanol EPH, and butyl carbitol, respectively. Only Solution 2, which employed Tamisolve NxG, was clear at the time of preparation. This solution remained clear after storage at room temperature for 24 hours and after one cycle of freezing and thawing. Solutions 1, and 3-6 were cloudy at the time of preparation. Solutions 3 and 4 also precipitated after storage at room temperature for 24 hours. Example 1 shows that not all solvents are effective at solubilizing salicylic acid at a concentration of about 10 wt. % in the above base solutions.

In Solution 2, the ratio of the amount of solvent (wt. %) to the amount of antimicrobial surfactants (wt. %) is between 1:1 and 1:2, calculated as follows:

$$= 18 [ Tamisolve NxG ] : ( 0.4 \times 19 ) [ Dowfax C 6 L ( about 40 \% active ) ] + ( 0.4 \times 4.3 ) [ Dowfax C 10 L ( about 40 \% active ) ] + 24.5 [ Biosoft S - 101 LS ( about 100 \% active ) ] = 18 : 7.6 + 1.72 + 24.5 = 18 : 33.82 = 1 : 1.88$$

Conversely, the ratio of the amount of antimicrobial surfactant (wt. %) to the amount of the amount of solvent (wt. %) is 1.88:1, which is between 1:1 and 2:1. The combined total of these ingredients is about 51.82 wt. %.

### Example 2—Solubility of Salicylic Acid in Other Solvent Solutions

Solutions 7-17 were prepared to evaluate the solubility of salicylic acid (sparing soluble antimicrobial agent) at 9 wt. % in a base solution employing different solvents. These solutions are summarized in Table 2 below.

TABLE 2

| | 7 | 8 | 9 | 10 | 11 | 12 |
|---|---|---|---|---|---|---|
| INGREDIENT | Wt. % | Wt. % | Wt. % | Wt. % | Wt. % | Wt. % |
| Rhodiasolv | 19 | — | — | — | — | — |
| PolarClean | | | | | | |
| Benzyl alcohol | | 19 | — | — | — | — |
| Propylene carbonate | | — | 19 | — | — | — |
| Cyrene | | — | — | 19 | — | — |
| Dimethyl succinate | | — | — | | 19 | — |
| Propylene glycol | | — | — | — | — | 19 |
| Salicylic acid | 9 | 9 | 9 | 9 | 9 | 9 |
| Pluronic L62 | 5 | 5 | 5 | 5 | 5 | 5 |
| Dowfax C6L | 19 | 19 | 19 | 19 | 19 | 19 |
| Dowfax C10L | 4.3 | 4.3 | 4.3 | 4.3 | 4.3 | 4.3 |
| Citric acid | 12 | 12 | 12 | 12 | 12 | 12 |
| Cobratec 35-G | 1 | 1 | 1 | 1 | 1 | 1 |
| Dequest 2010 | 1.2 | 1.2 | 1.2 | 1.2 | 1.2 | 1.2 |
| Biosoft S-101 LS | 24.5 | 24.5 | 24.5 | 24.5 | 24.5 | 24.5 |
| Lutropur MSA 100 | 5 | 5 | 5 | 5 | 5 | 5 |
| pH | <0.6 | <0.6 | <0.6 | <0.6 | <0.6 | <0.6 |
| Clarity, upon | Clear | Cloudy | Cloudy | Cloudy | Cloudy | Cloudy |
| prep: | | | | | | |
| Clarity, 24 hr | Clear | Precipitated | Precipitated | Precipitated | Precipitated | Precipitated |
| at room temp.: | | | | | | |

| | 13 | 14 | 15 | 16 | 17 |
|---|---|---|---|---|---|
| INGREDIENT | Wt. % | Wt. % | Wt. % | Wt. % | Wt. % |
| Butyl lactate | 19 | — | — | — | — |
| Glycol ether PM | — | 19 | — | — | — |
| Acetate | | | | | |
| Methoxytriglycol | — | — | 19 | — | — |
| Glycol ether DB | — | — | — | 19 | — |
| Mineral spirits | — | — | — | — | 19 |
| 3139 | | | | | |
| Salicylic acid | 9 | 9 | 9 | 9 | 9 |
| Pluronic L62 | 5 | 5 | 5 | 5 | 5 |
| Dowfax C6L | 19 | 19 | 19 | 19 | 19 |
| Dowfax C10L | 4.3 | 4.3 | 4.3 | 4.3 | 4.3 |
| Citric acid | 12 | 12 | 12 | 12 | 12 |
| Cobratec 35-G | 1 | 1 | 1 | 1 | 1 |
| Dequest 2010 | 1.2 | 1.2 | 1.2 | 1.2 | 1.2 |
| Biosoft S-101 LS | 24.5 | 24.5 | 24.5 | 24.5 | 24.5 |
| Lutropur MSA 100 | 5 | 5 | 5 | 5 | 5 |
| pH | <0.6 | <0.6 | <0.6 | <0.6 | <0.6 |
| Clarity, upon | Cloudy | Cloudy | Cloudy | Cloudy | Cloudy |
| prep: | | | | | |
| Clarity, 24 hr | Precipitated | Precipitated | Precipitated | Precipitated | Precipitated |
| at room temp.: | | | | | |

The solvents used in solutions 7-17 were Rhodiasolv PolarClean, benzyl alcohol, propylene carbonate, cyrene, dimethyl succinate, propylene glycol, butyl lactate, glycol ether PM acetate, methoxytriglycol, glycol ether DB, and mineral spirits 3139, respectively.

Except for Solution 7, all solutions were cloudy upon preparation and precipitated upon storage at room temperature for 24 hours. Solution 7 employed the solvent Rhodiasolv PolarClean and was clear both upon preparation and after storage for 24 hours at room temperature. Thus, not all solvents will solubilize salicylic acid at a concentration of about 9 wt. % in the above base solution.

In Solution 7, the ratio of the amount of antimicrobial surfactants (wt. %) to the amount of solvent (wt. %) is between 1:1 and 3:1. The combined total of these ingredients is between about 49.02 wt. % and about 52.82 wt. % depending on the solvent used, since the purity of the solvents are different, as shown in Table A.

### Example 3—Varying Ratio of Sparingly Soluble Antimicrobial Agent and Antimicrobial Surfactant

Solutions 18-22 consisting of a solvent (Tamisolve NxG), a sparingly soluble antimicrobial agent (salicylic acid), and an antimicrobial surfactant (Biosoft S-101 LS) were prepared and are summarized in Table 3.

TABLE 3

| | 18 | 19 | 20 | 21 | 22 |
|---|---|---|---|---|---|
| INGREDIENT | Wt. % | Wt. % | Wt. % | Wt. % | Wt. % |
| Tamisolve NxG | 23.54 | 23.54 | 23.54 | 23.54 | 23.54 |
| Salicylic acid | 20 | 18 | 16 | 14 | 13 |
| Biosoft S-101 LS | 56.46 | 58.46 | 60.46 | 62.46 | 63.46 |
| Solution pH | <0.6 | <0.6 | <0.6 | <0.6 | <0.6 |
| Clarity, upon prep: | Clear | Clear | Clear | Clear | Clear |
| Clarity, 24 hr at room | Precipitated | Precipitated | Precipitated | Precipitated | Clear |
| temp.: | | | | | |
| Clarity, freeze-thaw: | — | — | — | — | Clear |

Solutions 18-22 contained the same amount of solvent. The relative amounts of salicylic acid and anionic surfactant were varied as shown above. Of these solutions, only Solution 22 was both clear at the time of preparation and after storage at room temperature for 24 hours. Solution 22 was also clear after one cycle of freezing and thawing. The other solutions (Solutions 18-21) were clear at the time of preparation but precipitated after storage for 24 hours at room temperature. As such, they were not subjected to a cycle of freezing and thawing.

Example 3 shows that there is an upper limit to the amount of salicylic acid that can be stably solubilized in a solution containing only Tamisolve NxG and Biosoft S-101 LS, where the ratio of salicylic acid to anionic surfactant is varied.

In Solution 22, the ratio of the amount of antimicrobial surfactant (Biosoft S-101 LS) (wt. %) to the amount of the amount of solvent (Tamisolve NxG) (wt. %) is between 2:1 and 3:1 and the combined total of these ingredients is about 87 wt. %.

### Example 4—Concentrated Solution Embodiment

Concentrated Solution 23 was prepared using (among other things) a sparingly soluble antimicrobial agent (salicylic acid), antimicrobial surfactants (Biosoft S-101 LS and Dowfax C6L), and a solvent (Tamisolve NxG). The solution is summarized in Table 4 below.

TABLE 4

| | 23 |
|---|---|
| INGREDIENT | Wt. % |
| Tamisolve NxG | 19.7 |
| Salicylic acid | 11.7 |
| Biosoft S-101 LS | 24.8 |
| Dowfax C6L | 23.3 |
| Citric acid | 12 |
| Cobratec 35-G | 1 |
| p-Toluenesulfonic | 7.5 |
| acid | |
| Solution pH | <0.6 |
| Clarity, upon prep: | Clear |
| Clarity, 24 hr at room | Clear |
| temp.: | |
| Clarity, freeze-thaw: | Clear |

As shown above, Solution 23 was clear upon preparation, after storage for 24 hours at room temperature, and after one cycle of freezing and thawing.

In Solution 23, the ratio of the amount of antimicrobial surfactant (Biosoft S-101 LS and Dowfax C6L) (wt. %) to the amount of the amount of solvent (Tamisolve NxG) (wt. %) is between 1:1 and 2:1 and the combined total of these ingredients is about 53.82 wt. %.

### Example 4.1—Antimicrobial Efficacy of Diluted, RTU Version of Solution 23

Table 4.1 below shows virucidal testing results using solution 23 at different dilutions. Dilutions were made using hard water, and the virucidal tests were conducted using a modified GST method, wherein the steps of growth and enumeration of viruses were in accordance with the ASTM E1053 standard test method, and the antimicrobial efficacy testing steps were in accordance with the GST method. In this experiment, three different small non-enveloped viruses were used as shown below. Small non-enveloped viruses are known to be very tough to inactivate using disinfectants.

TABLE 4.1

| | 1:128 | 1:142 | 1:160 | 1:182 | 1:512 | 1:568 |
|---|---|---|---|---|---|---|
| | dilution | dilution | dilution | dilution | dilution | dilution |
| Canine Parvovirus | Pass (3 min) | Pass (3 min) | Pass (3 min) | Pass (3 min) | not tested | not tested |
| Feline | Pass (4 min) | Pass (4 min) | Pass (4 min) | Pass (4 min) | not tested | not tested |
| Panleukopenia | | | | | | |
| Virus | | | | | | |
| Poliovirus Type 1 | Pass (3 min) | Pass (3 min) | Pass (3 min) | Pass (3 min) | Pass (4 min) | Pass (4 min) |

Solution 23 at dilution ratios of 1:128 and as high as 1:568 was able to inactivate these hardy viruses at contact times less than 5 minutes (i.e., 4 minutes or less). Given these results, it is expected that Solution 23 will be effective to kill viruses such as poliovirus at a dilution ratio as high as 1:1136 at a contact time as short as 8 minutes.

### Example 5—Effect of pH on Antimicrobial Efficacy of Diluted, Ready-to-Use Version of Solution 23

Solution 23 was diluted with water at a ratio of 1:256 and the pH of the diluted solutions was varied by the addition of varying amounts of a pH adjusting agent, potassium hydroxide (KOH) to provide RTU Solutions 24-31. The solutions were evaluated for their antimicrobial efficacy against Staphylococcus aureus using the Germicidal Spray Testing (“GST”) AOAC 961.02 test method, at room temperature and a contact time of 5 or 10 minutes. The solutions and their antimicrobial test results are summarized in Table 5 below.

TABLE 5

| | 24 | 25 | 26 | 27 | 28 | 29 | 30 | 31 |
|---|---|---|---|---|---|---|---|---|
| pH | Unadjusted | pH to | pH to | pH to | pH to | pH to | pH to | pH to |
| adjustment | (pH 2.2) | 2.5 | 3.0 | 3.5 | 4.0 | 5.0 | 6.0 | 7.16 |
| using KOH | | | | | | | | |
| GST | Pass | Pass | Pass | Pass | Pass | Pass | Pass | Fail |
| Bactericidal | | | | | | | | |
| Efficacy | | | | | | | | |
| (S. aureus), | | | | | | | | |
| 10 minutes, | | | | | | | | |
| 5% soil load. | | | | | | | | |
| GST | Pass | Pass | Pass | Pass | Pass | Pass | Fail | Fail |
| Bactericidal | | | | | | | | |
| Efficacy | | | | | | | | |
| (S. aureus), | | | | | | | | |
| 5 minutes, | | | | | | | | |
| 5% soil load. | | | | | | | | |

Example 5 shows that the pH of the solution affects its antimicrobial efficacy. Preferably, embodiments of the invention have a pH up to about 6.0 (Solutions 24-30), more preferably up to about 5.0 (Solutions 24-29). Solution 31 which had a pH of about 7.16 failed the tests.

Since the pH of a solution increases when the solution is diluted with water, the skilled person will appreciate that the pH of concentrated versions of Solutions 24-31 will have even lower pH values than those specified above. As recited in Table 4, Solution 23 (prior to dilution) has a pH<0.6.

### Example 6—Solubility and Antimicrobial Activity of Different Sparingly Soluble Antimicrobial Agents

Solutions 32 to 35 were prepared using a different sparingly soluble antimicrobial agent and the same base solution. The solutions were observed to assess the solubility of the sparingly soluble antimicrobial agent in the base solution. The solutions were also diluted with water at a ratio of 1:256 (solution:water) and evaluated for their antimicrobial activity using the GST test method at room temperature and a contact time of 5 minutes. These solutions and the results are summarized in Table 6.

TABLE 6

| | 32 | 33 | 34 | 35 |
|---|---|---|---|---|
| INGREDIENT | Wt. % | Wt. % | Wt. % | Wt. % |
| Tamisolve NxG | 19.7 | 19.7 | 19.7 | 19.7 |
| Dowfax C6L | 23.3 | 23.3 | 23.3 | 23.3 |
| Biosoft S-101 LS | 24.8 | 24.8 | 24.8 | 24.8 |
| p-Toluenesulfonic acid | 7.5 | 7.5 | 7.5 | 7.5 |
| Benzoic acid | 11.7 | — | — | — |
| Thymol | — | 6 | — | — |
| Phenylacetic acid | — | — | 10 | — |
| Phenoxyacetic acid | — | — | — | 10 |
| pH | <0.6 | <0.6 | <0.6 | <0.6 |
| Clarity, upon prep: | Clear | Clear | Clear | Clear |
| Clarity, freeze-thaw: | Clear | Clear | Clear | Clear |
| GST Bactericidal Efficacy | Pass | Pass | Pass | Pass |
| (S. aureus), 1:256, 5 min, 5% soil | | | | |
| load. | | | | |

The above results show that the sparingly soluble antimicrobial agents, benzoic acid, thymol, phenylacetic acid, and phenoxyacetic acid, in Solutions 32-35 respectively, were fully soluble upon preparation and after a cycle of freezing and thawing.

When diluted at a ratio of 1:256 (solution:water), Solutions 32-35 also passed the GST method specified above.

For each of Solutions 32 to 35, the ratio of the amount of antimicrobial surfactant (Biosoft S-101 LS and Dowfax C6L) (wt. %) to the amount of the amount of solvent (Tamisolve NxG) (wt. %) is between 1:1 to 2:1 and the combined total of these ingredients is about 53.82 wt. %.

The skilled person will appreciate that different sparingly soluble antimicrobial agents will have different solubilities in an aqueous solution. The present invention is aimed at enhancing or increasing the solubility of the sparingly soluble antimicrobial agent relative to its solubility in water so that more concentrated solutions can be prepared to allow higher dilution ratios to be used prior to use.

### Example 7—Different Antimicrobial Surfactants

Solutions 36-41 were prepared to determine the solubility of 10 wt. % salicylic acid in a base solution comprising a selected antimicrobial surfactant, namely, Bioterge PAS-8S, Akypo LF 4, Calfoam ES-703, Texapon LS 30, Multitrope 1214, or Dowfax C10L. These solutions are summarized in Table 7 below.

TABLE 7

| | 36 | 37 | 38 | 39 | 40 | 41 |
|---|---|---|---|---|---|---|
| INGREDIENT | Wt. % | Wt. % | Wt. % | Wt. % | Wt. % | Wt. % |
| Tamisolve NxG | 21.5 | 21.5 | 21.5 | 21.5 | 21.5 | 21.5 |
| Salicylic acid | 10 | 10 | 10 | 10 | 10 | 10 |
| Bioterge PAS-8S | 61 | — | — | — | — | — |
| Akypo LF 4 | — | 35 | — | — | — | — |
| Calfoam ES-703 | — | — | 40 | — | — | — |
| Texapon LS 30 | — | — | — | 61 | — | — |
| Multitrope 1214 | — | — | — | — | 35 | — |
| Dowfax C10L | — | — | — | — | — | 61 |
| (40% active) | | | | | | |
| p-Toluenesulfonic | 7.5 | 7.5 | 7.5 | 7.5 | 7.5 | 7.5 |
| acid | | | | | | |
| pH | <0.6 | <0.6 | <0.6 | <0.6 | <0.6 | <0.6 |
| Clarity, upon | Clear | Clear | Clear | Clear | Clear | Clear |
| prep: | | | | | | |
| Clarity, 24 hr | Clear | Clear | Clear | Clear | Clear | Clear |
| at room temp.: | | | | | | |
| Clarity, | Clear | Clear | Clear | Clear | Clear | Clear |
| freeze-thaw: | | | | | | |

As shown above, Solutions 36-41 were all clear, including after storage at room temperature for 24 hours and after one cycle of freezing and thawing.

For each of Solutions 36 to 41, the ratio of the amount of antimicrobial surfactant (wt. %) to the amount of the amount of solvent (Tamisolve NxG) (wt. %) is between 2:1 to 1:2, respectively, and the combined total of these ingredients is in the range of about 38.58 wt. % to about 56.5 wt. %.

### Example 8—Varying Ratio of Sparingly Soluble Antimicrobial Agent to Solvent

Solution 42 was prepared and visually inspected at the time of preparation, after 24 hours of storage at room temperature, and after one cycle of freezing and thawing. The solution and results are summarized in Table 8 below.

TABLE 8

| | 42 |
|---|---|
| INGREDIENT | Wt. % |
| Tamisolve NxG | 35 |
| Salicylic acid | 20 |
| Biosoft S-101 LS | 45 |
| pH | <0.6 |
| Clarity, upon prep: | Clear |
| Clarity, 24 hr at room | Clear |
| temp.: | |
| Clarity, freeze-thaw: | Clear |

These results can be compared to the results of Table 3 wherein the maximum amount of the salicylic acid that was solubilized was 13 wt. %. Here, 20 wt. % salicylic acid was effectively solubilized by increasing the amount of the solvent (Tamisolve NxG) from 23.54 wt. % to 35 wt. % and decreasing the amount of the antimicrobial surfactant (Biosoft S-101 LS).

In this example, the solubility of salicylic acid is about 83 times its solubility in water. This example illustrates the greater contribution of the solvent compared to the antimicrobial surfactant to the solubility of the sparingly soluble antimicrobial agent.

Furthermore, for Solution 42, the ratio of the amount of antimicrobial surfactant (Biosoft S-101 LS) (wt. %) to the amount of the amount of solvent (Tamisolve NxG) (wt. %) is between 1:1 to 2:1, and the combined total of these ingredients is about 80 wt. %.

### Example 9—Additional Embodiments and Comparison Data

Additional concentrate Solutions 43, 44, and 45 were prepared and are summarized in Table 9 below.

TABLE 9

| | 43 | 44 | 45 |
|---|---|---|---|
| INGREDIENT | Wt. % | Wt. % | Wt. % |
| Rhodiasolv PolarClean | 21 | — | — |
| Dimethyl adipate | — | 21 | — |
| Dimethyl succinate | — | — | 21 |
| Salicylic acid | 10 | 10 | 10 |
| Phenylacetic acid | 1.5 | 1.5 | 1.5 |
| Biosoft S-101 LS | 25 | 25 | 25 |
| Lactic acid | 10 | 10 | 10 |
| Citric acid | — | — | — |
| Dowfax C10L | 25 | 25 | 25 |
| p-Toluenesulfonic acid | 7.5 | 7.5 | 7.5 |
| Cobratec ® 35-G | — | — | — |
| pH | <0.6 | <0.6 | <0.6 |
| Clarity, upon prep: | Clear | Cloudy | Cloudy |
| Clarity, freeze-thaw: | Clear | — | — |

As can be seen above, Solutions 43, 44 and 45 were identical except for the solvent employed (Rhodiasolv PolarClean, dimethyl adipate, or dimethyl succinate). Table 9 further illustrates that not all solvents work to solubilize relatively high amounts of sparingly soluble antimicrobial agents (salicylic acid and phenylacetic acid). Only Solution 43, which employed 21 wt. % of the solvent Rhodiasolv Polarclean, was effective to solubilize two sparingly soluble antimicrobial agents in a total concentration of 11.5 wt. %.

For Solution 43, the ratio of the amount of antimicrobial surfactants (Biosoft S-101 LS and Dowfax C10L) (wt. %) to the amount of the amount of solvent (Rhodiasolv PolarClean) (wt. %) is between 1:1 to 2:1, and the combined total of these ingredients is from about 51.8 wt. % to about 54.95 wt. % having regard to the purity of the ingredients shown in Table A.

### Example 10

Additional embodiments according to the invention were formulated and are summarized in Table 10 below.

TABLE 10

| | 46 | 47 | 48 |
|---|---|---|---|
| INGREDIENT | Wt. % | Wt. % | Wt. % |
| Rhodiasolv PolarClean | 23.4 | — | |
| Rhodiasolv Iris | — | 23 | |
| Tamisolve NxG | | | 19.7 |
| Salicylic acid | 8 | 8 | 11.7 |
| Phenylacetic acid | — | 1.5 | — |
| Biosoft S-101 LS | 24.8 | 25 | 24.8 |
| Lactic acid | — | 10 | |
| Citric acid | 12 | — | 12 |
| Dowfax C6L | — | — | 23.3 9.32 |
| Dowfax C10L | 23.3 | 25 10 | |
| p-Toluenesulfonic acid | 7.5 | 7.5 | 7.5 |
| Cobratec ® 35-G | 1 | — | 1 |
| pH | <0.6 | <0.6 | <0.6 |
| Clarity, upon prep: | Clear | Clear | Clear |
| Clarity, freeze-thaw: | Clear | Clear | Clear |

This example shows that the solvent Rhodiasolv Iris is also an effective solvent in the context of the present invention. Here, it was shown to effectively solubilize two sparingly soluble antimicrobial agents (salicylic acid and phenylacetic acid) in a base solution containing two antimicrobial surfactants (Biosoft S-101 LS with one of Dowfax C6L and Dowfax C10L) and no corrosion inhibitor (Cobratec 35-G).

For Solutions 46 to 48, the ratio of the amount of antimicrobial surfactants (Biosoft S-101 LS, Dowfax C6L or Dowfax C10L) (wt. %) to the amount of the amount of solvent (Rhodiasolv PolarClean, Rhodiasolv Iris, or Tamisolve NxG,) (wt. %) is between 1:1 to 2:1, respectively, and the combined total of these ingredients is from about 52.84 wt. % to about 58.04 wt. %, having regard to the purity of the ingredients shown in Table A.

### Example 11

Solutions 49 to 53 were prepared to determine the solubility of 10 wt. % salicylic acid in a base solution comprising one or two antimicrobial surfactants, namely, Dowfax C6L, Mackamine LO, Cocamidopropyl Betaine, Maprosyl 30-B+Biosoft S-101 LS, and Hostapon CT Paste+Biosoft S-101 LS, respectively. These solutions are summarized in Table 11 below.

TABLE 11

| | 49 | 50 | 51 | 52 | 53 |
|---|---|---|---|---|---|
| INGREDIENT | Wt. % | Wt. % | Wt. % | Wt. % | Wt. % |
| Tamisolve NxG | 21.5 | 21.5 | 21.5 | 21.5 | 21.5 |
| Salicylic acid | 10 | 10 | 10 | 10 | 10 |
| Tomadol 91-6 | — | 10 | 10 | — | — |
| Dowfax C6L | 61 | — | — | — | — |
| Biosoft S-101 LS | — | — | — | 12 | 12 |
| Mackamine LO | — | 51 | — | — | — |
| Cocamidopropyl Betaine | — | — | 51 | — | |
| Maprosyl 30-B | — | — | — | 49 | — |
| Hostapon CT Paste | — | — | — | — | 49 |
| p-Toluenesulfonic acid | 7.5 | 7.5 | 7.5 | 7.5 | 7.5 |
| pH | <0.6 | <0.6 | <0.6 | <0.6 | <0.6 |
| Clarity, upon prep: | Clear | Clear | Clear | Clear | Clear |
| Clarity, freeze-thaw: | Clear | Clear | Clear | Clear | Clear |

As shown above, Solutions 49 to 53 were all clear upon preparation and after one cycle of freezing and thawing.

In Solutions 49 to 53, the ratio of the amount of antimicrobial surfactant(s) (Dowfax C6L, Biosoft S-101 LS, Mackamine LO, Cocamidopropyl Betaine, Maprosyl 30-B, and Hostapon CT Paste) (wt. %) to the amount of the amount of solvent (Tamisolve NxG,) (wt. %) is between 2:1 and 1:2, and the combined total of these ingredients is from about 36.8 wt. % to about 48.2 wt. %.

### Solvent Information

The solvents that were used in the above experiments are summarized below in terms of their chemical classes, polarity, and proticity, which can directly impact the solvency attributes of each 5 solvent. The present invention requires at least one solvent that is an esteramide, diamide, branched dibasic ester, and/or alkyl pyrrolidone. These solvents are polar aprotic solvents. However, as shown by the experiments described above, not all polar aprotic solvents worked under the conditions of the tests. The non-working solvents, however, can be included as optional additional ingredients of solutions according to the invention.

TABLE 12

| Solvent | Solvent Class | Polarity | Proticity |
|---|---|---|---|
| Benzyl alcohol | Cyclic Alcohol | Polar | Protic |
| Butyl carbitol | Glycol Ether | Polar | Protic |
| Butyl lactate | Ester | Polar | Protic |
| Cyrene | Cycloalkanone | Polar | Aprotic |
| Dimethyl adipate | Dibasic Ester | Polar | Aprotic |
| Dimethyl succinate | Dibasic Ester | Polar | Aprotic |
| Dowanol EPH | Cyclic Alcohol | Polar | Protic |
| Dowanol TPM | Glycol Ether | Polar | Protic |
| Glycol ether DB | Glycol Ether | Polar | Protic |
| Glycol ether PM Acetate | Glycol Ether | Polar | Aprotic |
| Glycol ether PnP | Glycol Ether | Polar | Protic |
| Methoxytriglycol | Glycol Ether | Polar | Protic |
| Mineral spirits 3139 | Light Petroleum | Non-polar | Aprotic |
| | Oil | | |
| Propylene carbonate | Carbonate Ester | Polar | Aprotic |
| Propylene glycol | Dihydric Alcohol | Polar | Protic |
| Rhodiasolv PolarClean | Esteramide + | Polar | Aprotic |
| | Diamide | | |
| Rhodiasolve Iris | Branched Dibasic | Polar | Aprotic |
| | Ester | | |
| Tamisolve NxG | Alkyl Pyrrolidone | Polar | Aprotic |

### LIST OF ITEMS

The following is a non-exhaustive list of items provided by the invention.

Item 1. A phase stable, concentrated antimicrobial solution having a pH less than 0.6, comprising, consisting essentially of, or consisting of:
a. an effective amount of at least one sparingly soluble antimicrobial agent;
b. an effective amount of at least one antimicrobial surfactant selected from the group consisting of anionic surfactants and amphoteric surfactants; and
c. an effective amount of at least one solvent selected from the group consisting of esteramides, diamides, branched dibasic esters, and alkyl pyrrolidones;
wherein, the weight ratio of the at least one antimicrobial surfactant to the at least one solvent is from about 1:3 to about 3:1, and wherein the solution, when diluted with an aqueous diluent at a ratio of from about 1:128 to about 1:1024 has a pH of from about 1 to about 6, and passes the Germicidal Spray Testing AOAC 961.02 test method, at room temperature, and at a contact time of no greater than 10 minutes against S. aureus.

Item 2. The solution of item 1, wherein the at least one sparingly soluble antimicrobial agent is selected from the group consisting of phenolic compounds, C5-C28 carboxylic acids and salts thereof, terpenes, terpenoids, and essential oils.

Item 3. The solution of item 2, wherein the C5-C28 carboxylic acids and salts thereof are selected from the group consisting of hexanoic acid, isohexanoic acid, hexenoic acid, hydroxyhexanoic acid, heptanoic acid, isoheptanoic acid, hepteneoic acid, hydroxyheptanoic acid, octanoic acid, isooctanoic acid, octanoic acid, hydroxyoctanoic acid, decanoic acid, isodecanoic acid, hydroxydecanoic acid, dodecanoic acid, dodecenoic acid, isodecanoic acid, lauroyl-1-lactylic acid, lauroyl-2-lactylic acid, myristoyl-1-lactylic acid, myristoyl-2-lactylic acid, palmitoyl-1-lactylic acid, palmitoyl-2-lactylic acid, steroyl-2-lactylic acid, cocoyl sarcosine, myristoyl sarcosine, oleoyl sarcosine, C10-C28 alkyl ether carboxylic acids, benzoic acid, phenoxyacetic acid, phenylacetic acid, salicylic acid, hydroxybenzoic acid, dihydroxybenzoic acid, tropic acid, phthalic acid, terephthalic acid, methylbenzoic acid, anthranilic acid, furoic acid, cinnamic acid, hydroxycinnamic acid, picolinic acid, dipicolinic acid, pyridinedicarboxylic acid, pyridinecarboxylic acid, hydratropic acid, and salts thereof.

Item 4. The solution of item 3, wherein the C5-C28 carboxylic acids and salts thereof are selected from the group consisting of hexanoic acid, heptanoic acid, octanoic acid, nonanoic acid, decanoic acid, benzoic acid, phenoxyacetic acid, phenylacetic acid, salicylic acid, phthalic acid, furoic acid, cinnamic acid, and salts thereof.

Item 5. The solution of item 2, 3, or 4, wherein the antimicrobial essential oils, terpenes and terpenoids are selected from the group consisting of thymol, carvacrol, eugenol, linalool, linalyl acetate, citronellol, geraniol, cineol/eucalyptol, thujone, pinene, neral, borneol, terpenyl acetate, limonene, terpinene, methyl eugenol, acetyl eugenol, myrcene, terpinolene, terpineol, anisol, citronellal, neem oil and its extracts, tea tree oil and its extracts, neryl acetate, methyl cinnamate, geranyl acetate, and bergamottin.

Item 6. The solution of item 5, wherein the antimicrobial essential oils, terpenes and terpenoids are selected from the group consisting of thymol, carvacrol, eugenol, limonene, cinnamaldehyde, pinene, terpinene, terpinol, neem oil, tea tree oil, eucalyptus oil, eucalyptol, and anisole.

Item 7. The solution of any one of items 2 to 6, wherein the antimicrobial phenolic compounds are selected from the group consisting of salicylic acid, capryloyl salicylic acid, methylsalicylic acid, coumaric acid, ferulic acid, diferulic acid, sinapic acid, caffeic acid, protocatechuic acid, syringic acid, vanillic acid, gallic acid, anthranilic acid, ellagic acid, methyl phenol, dimethyl phenol, trimethyl phenol, diphenol, benzyloxy phenol, menthol, phenylphenol, hydroxyacetophenone, hydroxypropiophenone, hydroxyalkyl phenol, gingerol, naphthol, and naphthalenediol.

Item 8. The solution of item 7, wherein the antimicrobial phenolic compounds are selected from the group consisting of salicylic acid, capryloyl salicylic acid, caffeic acid, gallic acid, anthranilic acid, menthol, phenylphenol, and hydroxyacetophenone.

Item 9. The solution of item 1, wherein the at least one sparingly soluble antimicrobial agent is selected from the group consisting of salicylic acid, benzoic acid, thymol, carvacrol, gallic acid, limonene, phthalic acid, furoic acid, cinnamic acid, pinene, eucalyptol, neem oil, hexanoic acid, heptanoic acid, octanoic acid, nonanoic acid, and decanoic acid.

Item 10. The solution of any one of items 1 to 9, wherein the anionic surfactant is selected from the group consisting of C8-C28 sulfonates, C8-C28 sulfates, C8-C28 ether sulfates, C8-C28 ether sulfonates, C8-C28 olefin sulfonates, C8-C28 olefin sulfates, C6-C28 carboxylates, C6-C28 ether carboxylates, C8-C28 sulfonated carboxylates, C8-C28 phosphates, C8-C28 phosphonates, C8-C28 phosphate esters, C8-C28 taurates, C8-C24 amino acid based surfactants, and acid forms thereof.

Item 11. The solution of any one of items 1 to 10, wherein the amphoteric surfactant is selected from the group consisting of C8-C24 alkyl amine oxides, alkyl betaines, alkylamido sultaines, and amphocarboxylates.

Item 12. The solution of any one of items 1 to 11, wherein the esteramides are according to Formula I below:

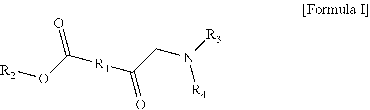

wherein R₁ is a C2-C8 branched or linear alkyl group;
R₂ is a C1-C6 linear, branched or cyclic alkyl group; and
R₃ and R₄ are independently methyl or ethyl.

Item 13. The solution of any one of items 1 to 12, wherein the diamides are according to Formula II:

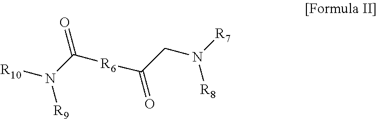

wherein R₆ is a C2-C8 branched or linear alkyl group; and
R₇, R₈, R₉ and R₁₀ are independently methyl or ethyl.

Item 14. The solution of any one of items 1 to 13, wherein the alkyl pyrrolidones are according to Formula III:

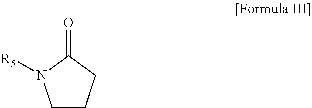

wherein R₅ is a branched or unbranched, saturated or unsaturated, substituted or unsubstituted C2 to C6 alkyl chain.

Item 15. The solution of item 14, wherein R₅ is a branched or unbranched, saturated or unsaturated, unsubstituted C3 to C5 alkyl chain or a C3 alkyl chain substituted with a methoxy group.

Item 16. The solution of any one of items 1 to 15, wherein the branched dibasic esters are according to Formula IV below:

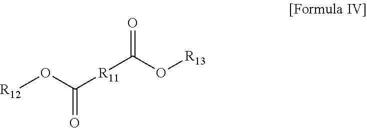

wherein R₁₁ is a C2-C8 branched alkyl group; and
R₁₂ and R₁₃ are independently a C1-C4 linear or branched alkyl group.

Item 17. The solution of any one of items 1 to 16, wherein the at least one antimicrobial surfactant and the at least one solvent are present at a combined total concentration of from at least 25 wt. %.

Item 18. The solution of item 17, wherein the at least one antimicrobial surfactant and the at least one solvent are present at a combined total concentration of from at least 40 wt. %.

Item 19. The solution of item 18, wherein the at least one antimicrobial surfactant and the at least one solvent are present at a combined total concentration of from at least 50 wt. %.

Item 20. The solution of item 19, wherein the at least one antimicrobial surfactant and the at least one solvent are present at a combined total concentration of from at least 60 wt. %.

Item 21. The solution of item 20, wherein the at least one antimicrobial surfactant and the at least one solvent are present at a combined total concentration of from at least 75 wt. %.

Item 22. The solution of any one of items 17 to 21, wherein the weight ratio of the at least one antimicrobial surfactant to the at least one solvent is from about 3:1 to about 1:2.

Item 23. The solution of item 22, wherein the weight ratio of the at least one antimicrobial surfactant to the at least one solvent is from about 2:1 to about 1:2.

Item 24. The solution of item 23, wherein the weight ratio of the at least one antimicrobial surfactant to the at least one solvent is from about 2:1 to about 1:1.

Item 25. The solution of any one of items 1 to 24, wherein the solubility of the at least one sparingly soluble antimicrobial agent is at least 20 times higher than its solubility in water.

Item 26. The solution of item 25, wherein the solubility of the at least one sparingly soluble antimicrobial agent is at least 30 times higher than its solubility in water.

Item 27. The solution of item 26, wherein the solubility of the at least one sparingly soluble antimicrobial agent is at least 40 times higher than its solubility in water.

Item 28. The solution of item 27, wherein the solubility of the at least one sparingly soluble antimicrobial agent is at least 50 times higher than its solubility in water.

Item 29. The solution of item 28, wherein the solubility of the at least one sparingly soluble antimicrobial agent is at least 60 times higher than its solubility in water.

Item 30. The solution of any one of items 1 to 29, further comprising an effective amount of at least one additional antimicrobial agent.

Item 31. The solution of item 30, wherein the at least one additional antimicrobial agent is selected from the group consisting of highly soluble antimicrobial carboxylic acids and salts thereof.

Item 32. The solution of item 31, wherein the highly soluble antimicrobial carboxylic acids and salts thereof are selected from the group consisting of acetic acid, citric acid, dimethylol propionic acid, formic acid, glutaric acid, glycolic acid, mandelic acid, lactic acid, maleic acid, malic acid, malonic acid, oxalic acid, propionic acid, succinic acid, sulfosuccinic acid, and salts thereof.

Item 33. The solution of item 32, wherein the highly soluble antimicrobial carboxylic acids and salts thereof are selected from the group consisting of citric acid, lactic acid, glycolic acid, mandelic acid, malic acid, oxalic acid, and salts thereof.

Item 34. The solution of any one of items 30 to 33 wherein the at least one additional antimicrobial agent is present in a concentration of from about 3 wt. % to about 30 wt. %.

Item 35. The solution of any one of items 1 to 34, wherein the at least one solvent is selected from the group consisting of N-butyl pyrrolidone, dimethyl 2-methylglutarate, pentanoic acid 5-(dimethylamino)-2-methyl-5-oxo-methyl ester and pentane-2-methyl-1,5-oxo-1,5-dimethylamine.

Item 36. The solution of any one of items 1 to 35, further comprising an effective amount of at least one optional functional ingredient selected from the group consisting of abrasive agents, additional solvents, additional surfactants, allergicides, anti-foaming agents, antioxidants, anti-pilling agents, anti-redeposition agents, anti-static agents, anti-wrinkling agents, buffering agents, builders, brightening agents, chelating agents, color protection agents, corrosion inhibitors, dyes, dye transfer inhibition agents, emulsifiers, enzymes, film forming agents, flame retardants, foaming agents, fragrances, hydrotropes, lubricants, metal salts, mildew removing agents, odor removal agents, odor capturing agents, peracid precursors, pH adjusting agents, plasticizers, preservatives, radical scavengers, rheology modifiers, skin conditioning agents, softening agents, soil releasing agents, soil shielding agents, soil suspenders, souring agents, stabilizing agents, ultraviolet light protection agents, vitamins, water repellency agents, and wound healing agents.

Item 37. The solution of item 36, wherein an effective amount of at least one acid pH adjusting agent is present.

Item 38. The solution of item 37, wherein the at least one acid pH adjusting agent has a pKa of less than 2.5 and is selected from the group consisting of methane sulfonic acid, toluene sulfonic acid, benzene sulfonic acid, sulfosalicylic acid, naphthalene disulfonic acid, sulfosuccinic acid, sulfuric acid, phosphoric acid, and sulfamic acid.

Item 39. The solution of item 36, 37, or 38, wherein an effective amount of at least one corrosion inhibitor is present.

Item 40. The solution of any one of items 1 to 39, wherein water is present, and the pH of the solution is from about-3 to about 1.

Item 41. The solution of item 40, wherein the pH of the solution is from about-2 to about 0.5.

Item 42. An RTU solution made by diluting a concentrated solution according to any one of items 1 to 41 with an aqueous diluent at a ratio of about 1:256 (solution:diluent), wherein the RTU solution has a pH of from about 1 to about 6 and passes the Germicidal Spray Testing AOAC 961.02 test method, at room temperature and a contact time of no greater than 10 minutes against S. aureus.

Item 43. An RTU solution made by diluting a concentrated solution according to any one of items 1 to 41 with an aqueous diluent at a ratio of about 1:128 (solution:diluent), wherein the RTU solution has a pH of from about 1 to about 6 and passes the Germicidal Spray Testing AOAC 961.02 test method, at room temperature and a contact time of no greater than 7 minutes against S. aureus.

Item 44. An RTU solution made by diluting a concentrated solution according to any one of items 1 to 41 with an aqueous diluent at a ratio of about 1:256 (solution:diluent), wherein the RTU solution has a pH of from about 1 to about 6 and passes the Germicidal Spray Testing AOAC 961.02 test method, at room temperature and a contact time of no greater than 7 minutes against S. aureus.

Item 45. An RTU solution made by diluting a concentrated solution according to any one of items 1 to 41 with an aqueous diluent at a ratio of about 1:128 (solution:diluent), wherein the RTU solution has a pH of from about 1 to about 6 and passes the Germicidal Spray Testing AOAC 961.02 test method, at room temperature and a contact time of no greater than 5 minutes against S. aureus.

Item 46. A kit of parts comprising parts packaged separately in at least two compartments and, wherein, when the parts are combined (optionally with the addition of an aqueous diluent), a solution according to any one of items 1 to 45 is formed.

Item 47. A method of reducing a microbial load on a surface contaminated with microbes, the method comprising:
d. identifying a surface contaminated with microbes;
e. providing a concentrated solution according to any one of items 1 to 41;
f. mixing the solution with an aqueous diluent at a ratio of from about 1:64 to about 1:1024 (solution:diluent) to produce a ready-to-use (RTU) solution;
g. applying an effective amount of the RTU solution to the surface for a contact of up to 10 minutes to reduce the microbial load on the surface.

Item 48. The method of item 47, wherein the surface is a hard surface, and the method is not a method of medical treatment.

Item 49. A method of making a concentrated antimicrobial solution containing at least one sparingly soluble antimicrobial agent in a concentration that exceeds its solubility in water by 20 to 60 times, the method comprising dissolving 20 to 60 times the amount of said at least one sparingly soluble antimicrobial agent than the amount that would dissolve in water in a solution comprising:
(a) an effective amount of at least one antimicrobial surfactant selected from the group consisting of anionic surfactants and amphoteric surfactants; and
(b) an effective amount at least one solvent selected from the group consisting of esteramides, diamides, branched dibasic esters, and alkyl pyrrolidones;
wherein, the weight ratio of the at least one antimicrobial surfactant to the at least one solvent is from about 1:3 to about 3:1, and optionally adding at least one functional ingredient to form a final solution, wherein the final solution has a pH less than 0.6.

Item 50. The solution of any one of items 17 to 21, wherein the weight ratio of the at least one antimicrobial surfactant to the at least one solvent is from about 1:1 to about 3:1.

Item 51. The solution of any one of items 17 to 21, wherein the weight ratio of the at least one antimicrobial surfactant to the at least one solvent is from about 1:1 to about 2:1.

Item 52. The solution of any one of items 17 to 21, wherein the weight ratio of the at least one antimicrobial surfactant to the at least one solvent is from about 1:1 to about 1:2.

Item 53. The solution of any one of items 17 to 21, wherein the weight ratio of the at least one antimicrobial surfactant to the at least one solvent is from about 1:1 to about 1:3.

Item 54. The solution of any one of items 17 to 21, wherein the weight ratio of the at least one antimicrobial surfactant to the at least one solvent is from about 1:2 to about 3:1.

Item 55. The solution of any one of items 17 to 21, wherein the weight ratio of the at least one antimicrobial surfactant to the at least one solvent is from about 1:2 to about 1:3.

Item 56. The solution of any one of items 17 to 21, wherein the weight ratio of the at least one antimicrobial surfactant to the at least one solvent is from about 3:1 to about 1:2.

Item 57. The solution of any one of items 17 to 21, wherein the weight ratio of the at least one antimicrobial surfactant to the at least one solvent is from about 2:1 to about 1:2.

Item 58. The solution of any one of items 17 to 21, wherein the weight ratio of the at least one antimicrobial surfactant to the at least one solvent is from about 3:1 to about 2:1.

The embodiments of the invention described herein are by way of example only and not intended to limit the scope of the invention as defined by the following claims.

1. A phase stable, concentrated antimicrobial solution having a pH less than 0.6, comprising:
a. an effective amount of at least one sparingly soluble antimicrobial agent selected from the group consisting of hexanoic acid, heptanoic acid, octanoic acid, nonanoic acid, decanoic acid, benzoic acid, phenoxyacetic acid, phenylacetic acid, salicylic acid, phthalic acid, furoic acid, cinnamic acid, thymol, carvacrol, eugenol, limonene, cinnamaldehyde, pinene, terpinene, terpinol, neem oil, tea tree oil, eucalyptus oil, eucalyptol, anisole, capryloyl salicylic acid, caffeic acid, gallic acid, anthranilic acid, menthol, phenylphenol, and hydroxyacetophenone;
b. an effective amount of at least one antimicrobial surfactant selected from the group consisting of C8-C28 sulfonates, C8-C28 sulfates, C8-C28 ether sulfates, C8-C28 ether sulfonates, C8-C28 olefin sulfonates, C8-C28 olefin sulfates, C6-C28 carboxylates, C6-C28 ether carboxylates, C8-C28 sulfonated carboxylates, C8-C28 phosphates, C8-C28 phosphonates, C8-C28 phosphate esters, C8-C28 taurates, C8-C24 amino acid based surfactants, and acid forms thereof; and C8-C24 alkyl amine oxides, alkyl betaines, alkylamido sultaines, and amphocarboxylates; and
c. an effective amount of at least one solvent selected from the group consisting of esteramides, diamides, branched dibasic esters, and alkyl pyrrolidones;
wherein the esteramides are according to Formula I below: 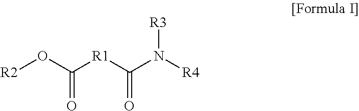
wherein R1 is a C2-C8 branched or linear alkyl group;
R2 is a C1-C6 linear, branched or cyclic alkyl group; and
R3 and R4 are independently methyl or ethyl;
wherein the branched dibasic esters are according to Formula IV below: 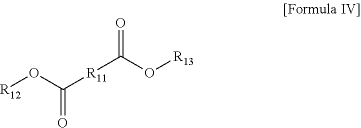
wherein R11 is a C2-C8 branched alkyl group; and
R12 and R13 are independently a C1-C4 linear or branched alkyl group;
wherein the diamides are according to Formula II: 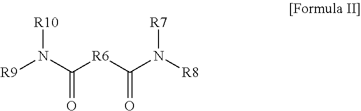
wherein R6 is a C2-C8 branched or linear alkyl group; and
R7, R8, R9 and R10 are independently methyl or ethyl;
wherein the alkyl pyrrolidones are according to Formula III: 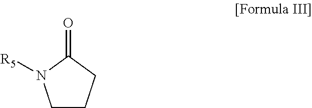
wherein R5 is a branched or unbranched, saturated or unsaturated, substituted or unsubstituted C2 to C6 alkyl chain; and
d. an effective amount of at least one acid pH adjusting agent having a pKa less than 2.3;
wherein, the weight ratio of the at least one antimicrobial surfactant to the at least one solvent is from about 1:3 to about 3:1, and wherein the solution, when diluted with an aqueous diluent at a ratio of from 1:128 to 1:1024 has a pH of from about 1 to about 6, and passes the Germicidal Spray Testing AOAC 961.02 test method, at room temperature, and at a contact time of no greater than 10 minutes against S. aureus.

2.-14. (canceled)

15. The solution of claim 1, wherein R₅ is a branched or unbranched, saturated or unsaturated, unsubstituted C3 to C5 alkyl chain or a C3 alkyl chain substituted with a methoxy group.

16. (canceled)

17. The solution of claim 1, wherein the at least one antimicrobial surfactant and the at least one solvent are present at a combined total concentration of from at least 25 wt. %.

18. The solution of claim 17, wherein the at least one antimicrobial surfactant and the at least one solvent are present at a combined total concentration of from at least 40 wt. %.

19. (canceled)

20. The solution of claim 18, wherein the at least one antimicrobial surfactant and the at least one solvent are present at a combined total concentration of from at least 60 wt. %.

21. (canceled)

22. The solution of claim 17, wherein the weight ratio of the at least one antimicrobial surfactant to the at least one solvent is from about 3:1 to about 1:2.

23. The solution of claim 22, wherein the weight ratio of the at least one antimicrobial surfactant to the at least one solvent is from about 2:1 to about 1:2.

24. The solution of claim 23, wherein the weight ratio of the at least one antimicrobial surfactant to the at least one solvent is from about 2:1 to about 1:1.

25.-29. (canceled)

30. The solution of claim 1, further comprising an effective amount of at least one additional antimicrobial agent.

31. The solution of claim 30, wherein the at least one additional antimicrobial agent is selected from the group consisting of highly soluble antimicrobial carboxylic acids and salts thereof.

32. (canceled)

33. The solution of claim 31, wherein the highly soluble antimicrobial carboxylic acids and salts thereof are selected from the group consisting of citric acid, lactic acid, glycolic acid, mandelic acid, malic acid, oxalic acid, and salts thereof.

34. The solution of claim 30, wherein the at least one additional antimicrobial agent is present in a concentration of from about 3 wt. % to about 30 wt. %.

35. The solution of claim 1, wherein the at least one solvent is selected from the group consisting of N-butyl pyrrolidone, dimethyl 2-methylglutarate, pentanoic acid 5-(dimethylamino)-2-methyl-5-oxo-methyl ester and pentane-2-methyl-1,5-oxo-1,5-dimethylamine.

36. The solution of claim 1, further comprising an effective amount of at least one optional functional ingredient selected from the group consisting of abrasive agents, additional solvents, additional surfactants, allergicides, anti-foaming agents, antioxidants, anti-pilling agents, anti-redeposition agents, anti-static agents, anti-wrinkling agents, buffering agents, builders, brightening agents, chelating agents, color protection agents, corrosion inhibitors, dyes, dye transfer inhibition agents, emulsifiers, enzymes, film forming agents, flame retardants, foaming agents, fragrances, hydrotropes, lubricants, metal salts, mildew removing agents, odor removal agents, odor capturing agents, peracid precursors, pH adjusting agents, plasticizers, preservatives, radical scavengers, rheology modifiers, skin conditioning agents, softening agents, soil releasing agents, soil shielding agents, soil suspenders, souring agents, stabilizing agents, ultraviolet light protection agents, vitamins, water repellency agents, and wound healing agents.

37. (canceled)

38. The solution of claim 1, wherein the at least one acid pH adjusting agent is selected from the group consisting of methane sulfonic acid, toluenesulfonic acid, benzenesulfonic acid, sulfosalicylic acid, naphthalene disulfonic acid, sulfosuccinic acid, sulfuric acid, phosphoric acid, and sulfamic acid.

39. The solution of claim 1, wherein an effective amount of at least one corrosion inhibitor is present.

40. The solution of claim 39, wherein the pH of the solution is from about-2 to about 0.5.

41. A ready-to-use (RTU) solution made by diluting a concentrated solution according to claim 1 with an aqueous diluent at a ratio of from about 1:128 to about 1:256 (solution:diluent).

42.-44. (canceled)

45. A method of reducing a microbial load on a surface contaminated with microbes, the method comprising:
a. identifying a surface contaminated with microbes;
b. providing a concentrated solution according to claim 1;
c. mixing the solution with an aqueous diluent at a ratio of from about 1:64 to about 1:1024 (solution:diluent) to produce a ready-to-use (RTU) solution;
d. applying an effective amount of the RTU solution to the surface for a contact of up to 10 minutes to reduce the microbial load on the surface.

46. The method of claim 45, wherein the surface is a hard surface, and the method is not a method of medical treatment.

47. (canceled)
